SEQUENCE LISTING (13 sequences)

SEQ ID NO: 1 - protein, 30 aa, Artificial Sequence
  Description of Artificial Sequence Synthetic polypeptide
PAICQRATATLGTVGSNTSGTTEIEACILL

SEQ ID NO: 2 - protein, 15 aa, Artificial Sequence
  Description of Artificial Sequence Synthetic peptide
IALILEPICCQERAA

SEQ ID NO: 3 - protein, 15 aa, Artificial Sequence
  Description of Artificial Sequence Synthetic peptide
IALILEPICCQERAA

SEQ ID NO: 4 - protein, 15 aa, Artificial Sequence
  Description of Artificial Sequence Synthetic peptide
  MOD_RES (8)..(8): Sarcosine
IALILEPXCCQERAA

SEQ ID NO: 5 - protein, 15 aa, Artificial Sequence
  Description of Artificial Sequence Synthetic peptide
  MOD_RES (9)..(9): Sarcosine
IALILEPIXCQERAA

SEQ ID NO: 6 - protein, 15 aa, Artificial Sequence
  Description of Artificial Sequence Synthetic peptide
  MOD_RES (3)..(3): D-amino acid
  MOD_RES (8)..(8): Sarcosine
IALILEPXCCQERAA

SEQ ID NO: 7 - protein, 15 aa, Artificial Sequence
  Description of Artificial Sequence Synthetic peptide
  MOD_RES (3)..(3): (R)-2-(7′-octenyl)alanine
  SITE (3)..(10): May or may not be stapled
  MOD_RES (8)..(8): Sarcosine
  MOD_RES (10)..(10): (S)-2-(4′-pentenyl)alanine
IAAILEPXCAQERAA

SEQ ID NO: 8 - protein, 15 aa, Artificial Sequence
  Description of Artificial Sequence Synthetic peptide
  MOD_RES (3)..(3): D-amino acid
  MOD_RES (8)..(8): Sarcosine
  MOD_RES (10)..(10): (S)-2-(4′-pentenyl)alanine
  SITE (10)..(14): May or may not be stapled
  MOD_RES (14)..(14): (S)-2-(4′-pentenyl)alanine
IALILEPXCAQERAA

SEQ ID NO: 9 - protein, 15 aa, Artificial Sequence
  Description of Artificial Sequence Synthetic peptide
  MOD_RES (7)..(7): (R)-2-(7′-octenyl)alanine
  SITE (7)..(14): May or may not be stapled
  MOD_RES (9)..(9): Sarcosine
  MOD_RES (14)..(14): (S)-2-(4′-pentenyl)alanine
IALILEAIXCQERAA

SEQ ID NO: 10 - protein, 15 aa, Artificial Sequence
  Description of Artificial Sequence Synthetic peptide
  MOD_RES (2)..(2): (S)-2-(4′-pentenyl)alanine
  SITE (2)..(6): May or may not be stapled
  MOD_RES (6)..(6): (S)-2-(4′-pentenyl)alanine
  MOD_RES (9)..(9): Sarcosine
IALILAPIXCQERAA

SEQ ID NO: 11 - protein, 15 aa, Artificial Sequence
  Description of Artificial Sequence Synthetic peptide
  MOD_RES (7)..(7): (S)-2-(4′-pentenyl)alanine
  SITE (7)..(11): May or may not be stapled
  MOD_RES (9)..(9): Sarcosine
  MOD_RES (11)..(11): (S)-2-(4′-pentenyl)alanine
IALILEAIXCAERAA

SEQ ID NO: 12 - protein, 15 aa, Artificial Sequence
  Description of Artificial Sequence Synthetic peptide
  MOD_RES (9)..(9): Sarcosine
  MOD_RES (10)..(10): D-amino acid
IALILEPIXCQERAA

SEQ ID NO: 13 - protein, 15 aa, Artificial Sequence
  Description of Artificial Sequence Synthetic peptide
  MOD_RES (9)..(9): Sarcosine
  MOD_RES (14)..(14): D-amino acid
IALILEPIXCQERAA

(19) United States
(12) Patent Application Publication
(10) Pub. No.: US 2023/0303623 A1
(43) Pub. Date: Sep. 28, 2023

(54) PEPTIDES AND METHODS OF USE

(71) Applicant: REALTA LIFE SCIENCES, INC., Norfolk, VA (US)

(72) Inventors: Neel K. KRISHNA, Norfolk, VA (US); Kenji CUNNION, Norfolk, VA (US); Ulrich THIENEL, Norfolk, VA (US)

(73) Assignee: REALTA LIFE SCIENCES, INC., Norfolk, VA (US)

(21) Appl. No.: 18/034,718

(22) PCT Filed: Nov. 1, 2021
(86) PCT No.: PCT/US2021/057495

§ 371 Date: May 1, 2023

Related U.S. Application Data
(60) Provisional application No. 63/108,762, filed on Nov. 2, 2020.

(51) Int. Cl. C07K 7/08; A61P 35/00

(52) U.S. Cl. CPC C07K 7/08; A61P 35/00

(57) ABSTRACT

The present invention provides synthetic peptides. The invention is directed to modifications of a synthetic peptide of 15 amino acids from the Polar Assonant (PA) peptide, which is a scrambled peptide derived from human astrovirus protein. In some embodiments, the invention is directed to peptides that are modifications of PA including sarcosine substitutions at certain amino acid positions that are stapled and/or have D-enantiomeric substitutions of certain amino acids. The invention further provides methods of selecting at least one synthetic peptide for treating various conditions.

### CROSS-REFERENCE TO RELATED APPLICATIONS

This application claims priority to U.S. Provisional Application No. 63/108,762, filed on 2 Nov. 2020, the disclosure of which is herein incorporated by reference in its entirety.

### SEQUENCE LISTING

The instant application contains a Sequence Listing which has been submitted electronically in ASCII format and is hereby incorporated by reference in its entirety. Said ASCII copy, created on Oct. 28, 2021, is named 251110 000156 SL.txt and is 5,875 bytes in size.

### BACKGROUND OF THE INVENTION

### 1. Field of the Invention

Embodiments of the present invention relates generally to synthetic peptides and uses thereof for therapy and diagnostics, and more specifically to stapled forms of the synthetic peptides, alone or in combination with D-enantiomers of certain amino acids of the synthetic peptides.

### 2. Background

The Complement System

The complement system, an essential component of the innate immune system, plays a critical role as a defense mechanism against invading pathogens, primes adaptive immune responses, and helps remove immune complexes and apoptotic cells. Three different pathways comprise the complement system: the classical pathway, the lectin pathway and alternative pathway. C1q and mannose-binding lectin (MBL) are the structurally related recognition molecules of the classical and lectin pathways, respectively. Whereas IgM or clustered IgG serve as the principal ligands for C1q, MBL recognizes polysaccharides such as mannan. Ligand binding by C1q and MBL results in the sequential activation of C4 and C2 to form the classical and lectin pathway C3-convertase, respectively. In contrast, alternative pathway activation does not require a recognition molecule, but can amplify C3 activation initiated by the classical or lectin pathways. Activation of any of these three pathways results in the formation of inflammatory mediators (C3a and C5a) and the membrane attack complex (MAC), which causes cellular lysis.

While the complement system plays a critical role in many protective immune functions, complement activation is a significant mediator of tissue damage in a wide range of autoimmune and inflammatory disease processes. (Ricklin and Lambris, “Complement-targeted therapeutics.” Nat Biotechnol 2007; 25(11):1265-75).

A need exists for complement regulators. On the one hand, the complement system is a vital host defense against pathogenic organisms. On the other hand, its unchecked activation can cause devastating host cell damage. Currently, despite the known morbidity and mortality associated with complement dysregulation in many disease processes, including autoimmune diseases such as systemic lupus erythematosus, myasthenia gravis, and multiple sclerosis, only two anti-complement therapies have recently been approved for use in humans: 1) eculizumab (Soliris™) and 2) ultomiris (Ravulizumab™) two humanized, long-acting monoclonal antibodies against C5 used in the treatment of paroxysmal nocturnal hemoglobinuria (PNH) and atypical hemolytic uremic syndrome (aHUS). PNH and aHUS are orphan diseases in which very few people are afflicted. Currently, no complement regulators are approved for the more common disease processes in which dysregulated complement activation plays a pivotal role. Dysregulated complement activation can play a role in both chronic disease indications and acute disease indications.

Developing peptides to inhibit classical, lectin and alternative pathways of the complement system is needed, as each of these three pathways have been demonstrated to contribute to numerous autoimmune and inflammatory disease processes. Specific blockade of classical and lectin pathways is particularly needed, as both of these pathways have been implicated in ischemia reperfusion-induced injury and other diseases in many animal models. Humans with alternative pathway deficiencies suffer severe bacterial infections. Thus, a functional alternative pathway is essential for immune surveillance against invading pathogens.

Naturally occurring peptides are essential signaling molecules that play critical physiological roles in human biology in the form of neurotransmitters, hormones, growth factors and anti-microbials [1]. Given their intrinsic specificity and efficient properties, this class of molecules have received considerable attention as human therapeutics for a variety of disease indications, with over 60 approved for therapeutic use in the US, Europe and/or Japan and 155 currently in clinical development as of March, 2018 [2]. The advantageous properties of peptides provides a significant advantage over small molecules (<500 Da) which often suffer from toxicity and off-target effects. Additionally, compared to large protein-based molecules such as humanized monoclonal antibodies, peptides typically enjoy low costs of manufacturing and in many cases can be synthesized chemically, thus avoiding costly and complex production and purification. Often, naturally occurring peptides cannot be directly translated into therapeutic use due to sub-optimal chemical and physical stability and poor pharmacokinetics (half-life). Thus, a number of technological approaches to rationally design peptides into more druggable molecules suitable for human administration are frequently employed.

The inventors have identified a novel family of peptides known as PIC1 (also referred to as EPICC peptides). The PIC1 peptides possess multiple anti-inflammatory properties including inhibition of the classical pathway of complement, myeloperoxidase (MPO) inhibition, neutrophil extracellular trap (NET) inhibition as well as intrinsic antioxidant and anti-microbial activity [3-8]. The precursor to the PIC1 peptides were initially based upon the finding that the 787 amino acid capsid protein sequence of human astrovirus type 1, a non-enveloped icosahedral RNA virus that is an endemic pathogen causing gastroenteritis in human infants [9], could inhibit activation of the classical pathway of complement [10].

The PIC1 family of molecules comprise a collection of rationally designed peptides with several anti-inflammatory functional properties including inhibition of the classical pathway of complement, myeloperoxidase inhibition, neutrophil extracellular trap inhibition and antioxidant activity. The original PIC1 peptide is a 15 amino acid peptide sequence, IALILEPICCQERAA (SEQ ID NO: 2), derived from a scrambled astroviral coat protein. The original PIC1 peptide has been modified with a C-terminal monodisperse 24-mer PEGylated moiety (IALILEPICCQERAA-dPEG24; PA-dPEG24; SEQ ID NO: 3), increasing its aqueous solubility. A sarcosine substitution scan of SEQ ID NO: 3 revealed that replacement of isoleucine at position 8 or cysteine at position 9 with sarcosine resulted in two peptides, IALILEP(Sar)CCQERAA (PA-I8Sar; SEQ ID NO: 4) and IALILEPI(Sar)CQERAA (PA-C9Sar; SEQ ID NO: 5), were water soluble without PEGylation (as described in U.S. Pat. No. 10,005,818). Additional variants based on the PA-I8Sar and PA-I9Sar molecules were constructed, including stapled forms of the peptides and/or one or more D-enantiomeric substitutions of certain amino acid positions.

### BRIEF SUMMARY OF THE INVENTION

As specified in the Background Section, there is a great need in the art to identify technologies for peptide-based inhibitors of the different pathways of the complement system and use this understanding to develop novel therapeutic peptides. The present invention satisfies this and other needs. Embodiments of the present invention relate generally to synthetic peptides and more specifically to synthetic peptides that are stapled and/or include one or more D-enantiomeric forms of the amino acids.

In one aspect, the present invention provides synthetic peptides that regulate the complement system and methods of using these peptides. Specifically, in some embodiments, the synthetic peptides can bind, regulate and inactivate C1 and MBL, and therefore can efficiently inhibit classical and lectin pathway activation at its earliest point while leaving the alternative pathway intact. These peptides are of therapeutic value for selectively regulating and inhibiting C1 and MBL activation without affecting the alternative pathway and can be used for treating diseases mediated by dysregulated activation of the classical and lectin pathways. In other embodiments, the peptides regulate classical pathway activation but not lectin pathway activation. The peptides are useful for various therapeutic indications.

In another aspect, the present invention provides synthetic peptides that inhibit programmed death-ligand 1 (PD-L1) binding to PD-1 receptor. PD-L1 is a 40 kDa type 1 transmembrane protein that suppresses the adaptive arm of immune system during particular events such as pregnancy, tissue allografts, autoimmune disease and other disease states. A number of human cancer cells express high levels of PD-L1, and blockade of this receptor reduces the growth of tumors in the presence of immune cells thus allowing tumor cells to evade anti-tumor immunity. PD-L1 acts as a checkpoint protein on myeloid cells and is a therapeutic target in cancer immunotherapy.

In some embodiments, the invention is based on the identification and modification of peptides of 15 amino acids from Polar Assortant (PA) peptide (SEQ ID NO: 2), modifications of the peptides, and methods of their use. The PA peptide is a scrambled peptide derived from human astrovirus protein, called CP1 (SEQ ID NO: 1). The PA peptide is also known as PIC1 (Peptide Inhibitors of Complement C1), AstroFend, AF, or SEQ ID NO: 2. The PIC1 peptide was originally named as such because it was found to be associated with diseases mediated by the complement system. A PEGylated form of the PIC1 peptide, called PA-dPEG24 (SEQ ID NO: 3), has 24 PEG moieties on the C-terminus of the peptide and was shown to have improved effects on complement inhibition. A form of the PIC1 peptide with the amino acid derivative sarcosine at position 8, called PA-I8Sar (SEQ ID NO: 4), also has improved effects on complement inhibition. A form of the PIC1 peptide with the amino acid derivative sarcosine at position 9, called PA-C9Sar (SEQ ID NO: 5), also has improved effects on complement inhibition. PA-dPEG24, PA-I8Sar, and PA-C9Sar are described in, e.g., U.S. Pat. No. 10,005,818, and U.S. Patent Publication No. US2019/0209660. As used herein, the term “PIC1 peptides” include SEQ ID NOs: 6-8 which are stapled forms of SEQ ID NO: 4 and/or substitutions of SEQ ID NO: 4 that have one or more D-enantiomeric forms of amino acids in place of the usual L-enantiomers, and SEQ ID NOs: 9-13 which are stapled forms of SEQ ID NO: 5 and/or substitutions of SEQ ID NO: 5 that have one or more D-enantiomeric forms of amino acids in place of the usual L-enantiomers.

In some aspects, the invention is directed to peptides that are stapled forms of PA-I8Sar and/or comprise one or more D-enantiomeric amino acid substitutions in the sequence of PA-I8Sar that are able to regulate the classical and lectin pathway activation by binding to C1q and MBL. In some aspects, the invention is directed to peptides that are stapled forms of PA-I9Sar and/or comprise one or more D-enantiomeric amino acid substitutions in the sequence of PA-I9Sar that are able to regulate the classical and lectin pathway activation by binding to C1q and MBL.

In some embodiments, the peptide sequence has at least about 95%, at least about 96%, at least about 97%, at least about 98%, or at least about 99% sequence identity to SEQ ID NOs: 6-13. In some embodiments, the peptide sequence has at least about 95%, at least about 96%, at least about 97%, at least about 98%, or at least about 99% sequence identity to SEQ ID NOs: 6-8. In some embodiments, the peptide sequence has at least about 95%, at least about 96%, at least about 97%, at least about 98%, or at least about 99% sequence identity to SEQ ID NOs: 9-13.

In one aspect, the invention provides a synthetic peptide comprising at least about 95% sequence identity to an amino acid sequence of SEQ ID NO: 6-8. In some embodiments, the invention is a synthetic peptide comprising the amino acid sequence and modifications of SEQ ID NO: 6-8. In one aspect, the invention is a synthetic peptide comprising at least about 95% sequence identity to an amino acid sequence of SEQ ID NO: 9-13. In some embodiments, the invention is a synthetic peptide comprising the amino acid sequence and modifications of SEQ ID NO: 9-13.

In another aspect, the invention provides a pharmaceutical composition comprising a therapeutically effective amount of at least one synthetic peptide selected from the group consisting of SEQ ID NO: 6-13, and variants thereof, and at least one pharmaceutically acceptable carrier, diluent, or excipient. In another aspect, the invention is a pharmaceutical composition comprising a therapeutically effective amount of at least one synthetic peptide selected from the group consisting of SEQ ID NO: 6-8, and variants thereof, and at least one pharmaceutically acceptable carrier, diluent, or excipient. In another aspect, the invention is a pharmaceutical composition comprising a therapeutically effective amount of at least one synthetic peptide selected from the group consisting of SEQ ID NO: 9-13, and variants thereof, and at least one pharmaceutically acceptable carrier, diluent, or excipient.

In one aspect, the invention provides a synthetic peptide comprising at least about 95% sequence identity to an amino acid sequence selected from the group of SEQ ID NO: 6-13.

In some embodiments, the invention provides a synthetic peptide comprising an amino acid sequence selected from the group consisting of SEQ ID NO: 6-13.

In a related aspect, the invention provides a pharmaceutical composition comprising a therapeutically effective amount of any of the synthetic peptides disclosed herein and at least one pharmaceutically acceptable carrier, diluent, or excipient.

In a related aspect, the invention provides a method of regulating the complement system comprising administering a pharmaceutical composition as described herein to a subject in need thereof.

In a related aspect, the invention provides a method of inhibiting myeloperoxidase activity comprising administering a pharmaceutical composition as described herein to a subject in need thereof.

In a related aspect, the invention provides a method of inhibiting oxidant activity comprising administering a pharmaceutical composition as described herein to a subject in need thereof.

In a related aspect, the invention provides a method of inhibiting binding of PD-1 and PD-L1 comprising administering a pharmaceutical composition as described herein to a subject in need thereof.

In a related aspect, the invention provides a method of inhibiting T cell exhaustion comprising administering a pharmaceutical composition as described herein to a subject in need thereof.

In a related aspect, the invention provides a method of inhibiting angiogenesis comprising administering a pharmaceutical composition as described herein to a subject in need thereof.

These and other objects, features and advantages of the present invention will become more apparent upon reading the following specification in conjunction with the accompanying description, claims and drawings.

### BRIEF DESCRIPTION OF THE DRAWINGS

The accompanying Figures, which are incorporated in and constitute a part of this specification, illustrate several aspects described below.

FIG. 1A-1B show PIC1 peptides' inhibition of complement activation in an ABO incompatibility assay. FIG. 1A shows modifications of PA-I8Sar and FIG. 1B shows modifications of PA-C9Sar. Inhibition of ABO incompatibility hemolysis in a CH50-type assay. Peptides are at a final concentration of 0.5 mM. Values are represented as a percent of the positive control, which consists of human 0 sera and AB red blood cells in GVBS⁺⁺ buffer. Data are the means of n=3 independent experiments±SEM.

FIG. 2A-2B show half-maximal binding values for PIC1 peptides binding to C1q. Half-maximal binding concentrations were calculated for (2A) PA-I8Sar and (2B) PA-I9Sar variants from the binding curves. Peptide variant PA-0142 did not bind C1q whereas peptide variant PA-0152 did not titrate so a half-maximal binding could not be calculated and peptide variant PA-0168 could not be analyzed as it was not recognized by the primary polyclonal antibody.

FIG. 3A-3B show half-maximal values for PIC1 peptides inhibition of MPO activity. Half-maximal values were calculated for (3A) PA-I8Sar and (3B) PA-C9Sar variants from the activity curves.

FIG. 4A-4B show PIC1 peptide inhibition of oxidant activity in a Total Antioxidant Capacity (TAC) assay. Antioxidant activity is measured in copper reducing equivalents (CRE). (4A) PA-I8Sar and (4B) PA-I9Sar variants were tested over a range of concentrations and the maximal amounts of antioxidant activity is reported for each peptide.

FIG. 5A-5C show C1q binding curves. Binding of increasing concentrations of PA-I8Sar modifications (5A-5B) and PA-I9Sar modifications (5C) to immobilized C1q in an ELISA-type assay. Peptide variant PA-0142 did not bind C1q whereas peptide variant PA-0152 did not titrate so a half-maximal binding could not be calculated and peptide variant PA-0168 could not be analyzed as it was not recognized by the primary polyclonal antibody.

FIG. 6A-6C show MPO inhibition curves. Inhibition of MPO activity by increasing concentrations of PA-I8Sar modifications (6A-6B) and PA-I9Sar modifications (6C) in an ELISA-type assay.

FIG. 7A-7C show total antioxidant capacity activity curves. Increasing concentrations of PA-I8Sar modifications (7A-7B) and PA-I9Sar modifications (7C) were analyzed for total antioxidant activity. Antioxidant activity is measured in copper reducing equivalents (CRE).

FIG. 8 shows inhibition of PD-1 binding to PD-L1 in an ELISA plate-based assay. PIC1 peptides were allowed to bind to immobilized PD-L1 on the surface of the plate. Biotinylated PD-1 was then added and bound PD-1 detected by streptavidin-HRP reagent followed by TMB as the substrate for the colorimetric assay.

FIG. 9A-9B shows RLS-0134 (9A) and RLS-0150 (9B) binding to CTLA-4, PD-1 and PD-L1 in an ELISA plate-based assay. RLS-0134 and RLS-0150 were allowed to bind to immobilized CTLA-4, PD-1, PD-L1 on the surface of the plate. Bound C1q and MAC-1 served as a positive and negative control for peptide binding, respectively. Increasing amounts of peptides were added to the plates followed by a rabbit polyclonal antibody that recognizes the peptides and then a secondary anti-rabbit antibody conjugated with HRP. The plate was then developed by addition of TMB as the substrate for the colorimetric assay.

FIG. 10 shows that RLS-0122, RLS-0150, RLS-0154, RLS-0164 and RLS-0168 were able to inhibit CTLA-4 mediated cell signaling. CTLA-4 effector cells were incubated with aAPC/Raji Cells in the absence or presence of increasing concentrations of anti-CTLA-4 antibody (positive control), RLS-0122, RLS-0150, RLS-0154, RLS-0164 and RLS-0168 and RLS-0088 (negative control). Luminescence was detected in luminometer plate reader. The drop-off in signal with the test peptides at high concentrations is due to cell death as a result of peptide buffer effects on the cells.

FIG. 11 shows that RLS-0122, RLS-0164 and RLS-0168 were able to inhibit T cell exhaustion as measured by reduced levels of apoptotic cell markers Caspase 3/7. Purified human pan-T-cells were subject to stimulation with Dynabeads every 48 hours over the period of 8 days with cells also receiving PIC1 peptides (2 mg/ml) at each stimulation. Cells not receiving Dynabeads were run in parallel to assess background levels of Caspase 3/7 signal. At the eighth day, cells were harvested, and the levels of Caspase 3/7 determined by ELISA. RLS-0150 and RLS-0154 did not display reduction of Caspase levels.

FIG. 12A-12D shows that that RLS-0122, RLS-0150, RLS-0154, RLS-0164 and RLS-0168 were able to reverse T cell exhaustion as measured by increased levels of cytokines IL-2 (12A and 12C) and IFN-gamma (12B and 12D). RLS-0164 is shown in FIGS. 12A and B and RLS-0122, RLS-0150, RLS-0154, and RLS-0168 are shown in FIGS. 12C and 12D. Purified human pan-T-cells were subject to stimulation with Dynabeads every 48 hours over the period of 8 days with cells also receiving PIC1 peptides (2 mg/ml) at each stimulation. Cell supernatants were collected at each stimulation and levels of IL-2 and IFN-gamma assayed by ELISA.

FIG. 13 shows PIC1 peptides binding to VEGF in an ELISA plate-based assay. PIC1 peptides were allowed to bind to immobilized VEGF on the surface of the plate. A fixed amount of PIC1 peptides (1 mg/ml) were added to the plates followed by a rabbit polyclonal antibody that recognizes the peptides and then a secondary anti-rabbit antibody conjugated with HRP. The plate was then developed by addition of TMB as the substrate for the colorimetric assay.

FIG. 14 shows that specific PIC1 peptides were able to inhibit VEGF mediated cell signaling. VEGF effector cells were incubated with RLS-0122, RLS-0150, RLS-0154, RLS-0164 and RLS-0168 and then VEGF was added. Luminescence was detected in luminometer plate reader. Cells incubated with VEGF alone was a positive control for VEGF-mediated cell signaling depicted by the line showing 20,000 relative luminescence units (RLS) response indicative of VEGF binding VEGFR-2.

FIG. 15 shows that specific PIC1 peptides were able to inhibit non-VEGF mediated angiogenesis induced by LPS. HUVEC cells were incubated with either RLS-0122, RLS-0150, RLS-0154, RLS-0164 and RLS-0168 followed by LPS addition and plating onto extracellular matrix. After overnight incubation, evidence of angiogenesis was determined by fluorescence microscopy. Cells receiving LPS and not receiving LPS served as positive and negative controls for angiogenesis.

FIG. 16 shows that RLS-0122 can inhibit classical complement pathway activation by human kidney carcinoma cell line A498. Supernatants from A-498 cells were added to purified human C1q pre-incubated with increasing doses of RLS-0122 or RLS-0174 and loaded onto an IgG coated plate. Samples were then incubated for 1 hour at 37° C. After incubation samples were washed 3 times with PBS-T before the addition of purified human C4 (4 ug/ml) and incubated for 1.5 hours at 37° C. The supernatants were collected for analysis on Quidel's MicroVue Complement C4a ELISA.

FIG. 17A-17B shows the effects of RLS-0122 on survival (17A) and quality of life (17B) in a TC-1 cancer cell model in mice. Animals were injected with 4×10\hat{ }5 TC-1 cells subcutaneously into the flank of the animal. Five days later, animals received vehicle treatment with saline (n=6), or drug treatment with RLS-0122 (n=8) IV at 160 mg/kg 1×/day for 15 consecutive days. Animals were also evaluated for behavior and body condition scoring every 2 days for duration of the study. If scoring reached 7 or below, animals were assessed daily. A score of 5 or below was considered endpoint for euthanasia.

### DETAILED DESCRIPTION OF THE INVENTION

As specified in the Background Section, there is a great need in the art to identify technologies for peptide-based inhibitors of the different pathways of the complement system and use this understanding to develop novel therapeutic peptides. The present invention satisfies this and other needs. Embodiments of the present invention relate generally to synthetic peptides and more specifically to synthetic peptides that are stapled and/or include one or more D-enantiomeric forms of the amino acids.

To facilitate an understanding of the principles and features of the various embodiments of the invention, various illustrative embodiments are explained below. Although exemplary embodiments of the invention are explained in detail, it is to be understood that other embodiments are contemplated. Accordingly, it is not intended that the invention is limited in its scope to the details of construction and arrangement of components set forth in the following description or examples. The invention is capable of other embodiments and of being practiced or carried out in various ways. Also, in describing the exemplary embodiments, specific terminology will be resorted to for the sake of clarity.

It must also be noted that, as used in the specification and the appended claims, the singular forms “a,” “an” and “the” include plural references unless the context clearly dictates otherwise. For example, reference to a component is intended also to include composition of a plurality of components. References to a composition containing “a” constituent is intended to include other constituents in addition to the one named. In other words, the terms “a,” “an,” and “the” do not denote a limitation of quantity, but rather denote the presence of “at least one” of the referenced item.

As used herein, the term “and/or” may mean “and,” it may mean “or,” it may mean “exclusive-or,” it may mean “one,” it may mean “some, but not all,” it may mean “neither,” and/or it may mean “both.” The term “or” is intended to mean an inclusive “or.”

Also, in describing the exemplary embodiments, terminology will be resorted to for the sake of clarity. It is intended that each term contemplates its broadest meaning as understood by those skilled in the art and includes all technical equivalents which operate in a similar manner to accomplish a similar purpose. It is to be understood that embodiments of the disclosed technology may be practiced without these specific details. In other instances, well-known methods, structures, and techniques have not been shown in detail in order not to obscure an understanding of this description. References to “one embodiment,” “an embodiment,” “example embodiment,” “some embodiments,” “certain embodiments,” “various embodiments,” etc., indicate that the embodiment(s) of the disclosed technology so described may include a particular feature, structure, or characteristic, but not every embodiment necessarily includes the particular feature, structure, or characteristic. Further, repeated use of the phrase “in one embodiment” does not necessarily refer to the same embodiment, although it may.

As used herein, the term “about” should be construed to refer to both of the numbers specified as the endpoint (s) of any range. Any reference to a range should be considered as providing support for any subset within that range. Ranges may be expressed herein as from “about” or “approximately” or “substantially” one particular value and/or to “about” or “approximately” or “substantially” another particular value. When such a range is expressed, other exemplary embodiments include from the one particular value and/or to the other particular value. Further, the term “about” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Similarly, as used herein, “substantially free” of something, or “substantially pure”, and like characterizations, can include both being “at least substantially free” of something, or “at least substantially pure”, and being “completely free” of something, or “completely pure”.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

Throughout this description, various components may be identified having specific values or parameters, however, these items are provided as exemplary embodiments. Indeed, the exemplary embodiments do not limit the various aspects and concepts of the present invention as many comparable parameters, sizes, ranges, and/or values may be implemented. The terms “first,” “second,” and the like, “primary,” “secondary,” and the like, do not denote any order, quantity, or importance, but rather are used to distinguish one element from another.

It is noted that terms like “specifically,” “preferably,” “typically,” “generally,” and “often” are not utilized herein to limit the scope of the claimed invention or to imply that certain features are critical, essential, or even important to the structure or function of the claimed invention. Rather, these terms are merely intended to highlight alternative or additional features that may or may not be utilized in a particular embodiment of the present invention. It is also noted that terms like “substantially” and “about” are utilized herein to represent the inherent degree of uncertainty that may be attributed to any quantitative comparison, value, measurement, or other representation.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “50 mm” is intended to mean “about 50 mm.”

It is also to be understood that the mention of one or more method steps does not preclude the presence of additional method steps or intervening method steps between those steps expressly identified. Similarly, it is also to be understood that the mention of one or more components in a composition does not preclude the presence of additional components than those expressly identified.

The materials described hereinafter as making up the various elements of the present invention are intended to be illustrative and not restrictive. Many suitable materials that would perform the same or a similar function as the materials described herein are intended to be embraced within the scope of the invention. Such other materials not described herein can include, but are not limited to, materials that are developed after the time of the development of the invention, for example. Any dimensions listed in the various drawings are for illustrative purposes only and are not intended to be limiting. Other dimensions and proportions are contemplated and intended to be included within the scope of the invention.

As used herein, the term “subject” or “patient” refers to mammals and includes, without limitation, human and veterinary animals. In a preferred embodiment, the subject is human.

As used herein, the term “combination” of a synthetic peptide according to the claimed invention and at least a second pharmaceutically active ingredient means at least two, but any desired combination of compounds can be delivered simultaneously or sequentially (e.g., within a 24 hour period). It is contemplated that when used to treat various diseases, the compositions and methods of the present invention can be utilized with other therapeutic methods/agents suitable for the same or similar diseases. Such other therapeutic methods/agents can be co-administered (simultaneously or sequentially) to generate additive or synergistic effects. Suitable therapeutically effective dosages for each agent may be lowered due to the additive action or synergy.

A “disease” is a state of health of a subject wherein the subject cannot maintain homeostasis, and wherein if the disease is not ameliorated then the subject's health continues to deteriorate. In contrast, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health.

The terms “treat” or “treatment” of a state, disorder or condition include: (1) preventing or delaying the appearance of at least one clinical or sub-clinical symptom of the state, disorder or condition developing in a subject that may be afflicted with or predisposed to the state, disorder or condition but does not yet experience or display clinical or subclinical symptoms of the state, disorder or condition; or (2) inhibiting the state, disorder or condition, i.e., arresting, reducing or delaying the development of the disease or a relapse thereof (in case of maintenance treatment) or at least one clinical or sub-clinical symptom thereof; or (3) relieving the disease, i.e., causing regression of the state, disorder or condition or at least one of its clinical or sub-clinical symptoms. The benefit to a subject to be treated is either statistically significant or at least perceptible to the patient or to the physician.

The term “therapeutic” as used herein means a treatment and/or prophylaxis. A therapeutic effect is obtained by suppression, diminution, remission, or eradication of a disease state.

As used herein the term “therapeutically effective” applied to dose or amount refers to that quantity of a compound or pharmaceutical composition that when administered to a subject for treating (e.g., preventing or ameliorating) a state, disorder or condition, is sufficient to effect such treatment. The “therapeutically effective amount” will vary depending on the compound or bacteria or analogues administered as well as the disease and its severity and the age, weight, physical condition and responsiveness of the mammal to be treated.

The phrase “pharmaceutically acceptable”, as used in connection with compositions of the invention, refers to molecular entities and other ingredients of such compositions that are physiologically tolerable and do not typically produce untoward reactions when administered to a mammal (e.g., a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans.

The terms “pharmaceutical carrier” or “pharmaceutically acceptable carrier” refer to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Alternatively, the pharmaceutical carrier can be a solid dosage form carrier, including but not limited to one or more of a binder (for compressed pills), a glidant, an encapsulating agent, a flavorant, and a colorant. Suitable pharmaceutical carriers are described in “Remington's Pharmaceutical Sciences” by E. W. Martin.

The term “analog” or “functional analog” refers to a related modified form of a polypeptide, wherein at least one amino acid substitution, deletion, or addition has been made such that said analog retains substantially the same biological activity as the unmodified form, in vivo and/or in vitro.

The terms “sequence identity” and “percent identity” are used interchangeably herein. For the purpose of this invention, it is defined here that in order to determine the percent identity of two amino acid sequences or two nucleic acid sequences, the sequences are aligned for optimal comparison purposes (e.g., gaps can be introduced in the sequence of a first amino acid or nucleic acid for optimal alignment with a second amino or nucleic acid sequence). The amino acid or nucleotide residues at corresponding amino acid or nucleotide positions are then compared. When a position in the first sequence is occupied by the same amino acid or nucleotide residue as the corresponding position in the second sequence, then the molecules are identical at that position. The percent identity between the two sequences is a function of the number of identical positions shared by the sequences (i.e., % identity=number of identical positions/total number of positions (i.e., overlapping positions)×100). Preferably, the two sequences are the same length.

Several different computer programs are available to determine the degree of identity between two sequences. For instance, a comparison of sequences and determination of percent identity between two sequences can be accomplished using a mathematical algorithm. In a preferred embodiment, the percent identity between two amino acid or nucleic acid sequences is determined using the Needleman and Wunsch (J. Mol. Biol. (48): 444-453 (1970)) algorithm which has been incorporated into the GAP program in the Accelrys GCG software package (available at www.accelrys.com/products/gcg), using either a Blosum 62 matrix or a PAM250 matrix, and a gap weight of 16, 14, 12, 10, 8, 6, or 4 and a length weight of 1, 2, 3, 4, 5, or 6. These different parameters will yield slightly different results but the overall percentage identity of two sequences is not significantly altered when using different algorithms.

A sequence comparison may be carried out over the entire lengths of the two sequences being compared or over fragments of the two sequences. Typically, the comparison will be carried out over the full length of the two sequences being compared. However, sequence identity may be carried out over a region of, for example, twenty, fifty, one hundred or more contiguous amino acid residues.

“Sequence identity” as it is known in the art refers to a relationship between two or more polypeptide sequences or two or more polynucleotide sequences, namely a reference sequence and a given sequence to be compared with the reference sequence. Sequence identity is determined by comparing the given sequence to the reference sequence after the sequences have been optimally aligned to produce the highest degree of sequence similarity, as determined by the match between strings of such sequences. Upon such alignment, sequence identity is ascertained on a position-by-position basis, e.g., the sequences are “identical” at a particular position if at that position, the nucleotides or amino acid residues are identical. The total number of such position identities is then divided by the total number of nucleotides or residues in the reference sequence to give % sequence identity. Sequence identity can be readily calculated by known methods, including but not limited to, those described in Computational Molecular Biology, Lesk, A. N., ed., Oxford University Press, New York (1988), Biocomputing: Informatics and Genome Projects, Smith, D. W., ed., Academic Press, New York (1993); Computer Analysis of Sequence Data, Part I, Griffin, A. M., and Griffin, H. G., eds., Humana Press, New Jersey (1994); Sequence Analysis in Molecular Biology, von Heinge, G., Academic Press (1987); Sequence Analysis Primer, Gribskov, M. and Devereux, J., eds., M. Stockton Press, New York (1991); and Carillo, H., and Lipman, D., SIAM J. Applied Math., 48: 1073 (1988), the teachings of which are incorporated herein by reference. Preferred methods to determine the sequence identity are designed to give the largest match between the sequences tested. Methods to determine sequence identity are codified in publicly available computer programs which determine sequence identity between given sequences. Examples of such programs include, but are not limited to, the GCG program package (Devereux, J., et al., Nucleic Acids Research, 12(1):387 (1984)), BLASTP, BLASTN and FASTA (Altschul, S. F. et al., J. Molec. Biol., 215:403-410 (1990). The BLASTX program is publicly available from NCBI and other sources (BLAST Manual, Altschul, S. et al., NCVI NLM NIH Bethesda, Md. 20894, Altschul, S. F. et al., J. Molec. Biol., 215:403-410 (1990), the teachings of which are incorporated herein by reference). These programs optimally align sequences using default gap weights in order to produce the highest level of sequence identity between the given and reference sequences. As an illustration, by a polynucleotide having a nucleotide sequence having at least, for example, 95%, e.g., at least 96%, 97%, 98%, 99%, or 100% “sequence identity” to a reference nucleotide sequence, it is intended that the nucleotide sequence of the given polynucleotide is identical to the reference sequence except that the given polynucleotide sequence may include up to 5, 4, 3, 2, 1, or 0 point mutations per each 100 nucleotides of the reference nucleotide sequence. In other words, in a polynucleotide having a nucleotide sequence having at least 95%, e.g., at least 96%, 97%, 98%, 99%, or 100% sequence identity relative to the reference nucleotide sequence, up to 5%, 4%, 3%, 2%, 1%, or 0% of the nucleotides in the reference sequence may be deleted or substituted with another nucleotide, or a number of nucleotides up to 5%, 4%, 3%, 2%, 1%, or 0% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence. Analogously, by a polypeptide having a given amino acid sequence having at least, for example, 95%, e.g., at least 96%, 97%, 98%, 99%, or 100% sequence identity to a reference amino acid sequence, it is intended that the given amino acid sequence of the polypeptide is identical to the reference sequence except that the given polypeptide sequence may include up to 5, 4, 3, 2, 1, or 0 amino acid alterations per each 100 amino acids of the reference amino acid sequence. In other words, to obtain a given polypeptide sequence having at least 95%, e.g., at least 96%, 97%, 98%, 99%, or 100% sequence identity with a reference amino acid sequence, up to 5%, 4%, 3%, 2%, 1%, or 0% of the amino acid residues in the reference sequence may be deleted or substituted with another amino acid, or a number of amino acids up to 5%, 4%, 3%, 2%, 1%, or 0% of the total number of amino acid residues in the reference sequence may be inserted into the reference sequence. These alterations of the reference sequence may occur at the amino or the carboxy terminal positions of the reference amino acid sequence or anywhere between those terminal positions, interspersed either individually among residues in the reference sequence or in the one or more contiguous groups within the reference sequence. Preferably, residue positions which are not identical differ by conservative amino acid substitutions. However, conservative substitutions are not included as a match when determining sequence identity.

As used herein, the term “immune response” includes innate immune responses as well as T-cell mediated and/or B-cell mediated immune responses. Exemplary immune responses include T cell responses, e.g., cytokine production and cellular cytotoxicity, and B cell responses, e.g., antibody production. In addition, the term “immune response” includes immune responses that are indirectly affected by T cell activation, e.g., antibody production (humoral responses) and activation of cytokine responsive cells, e.g., macrophages. Immune cells involved in the immune response include lymphocytes, such as B cells and T cells (CD4+, CD8+, Th1 and Th2 cells); antigen presenting cells (e.g., professional antigen presenting cells such as dendritic cells, macrophages, B lymphocytes, Langerhans cells, and non-professional antigen presenting cells such as keratinocytes, endothelial cells, astrocytes, fibroblasts, oligodendrocytes); natural killer cells; myeloid cells, such as macrophages, eosinophils, mast cells, basophils, and granulocytes (e.g. neutrophils).

“Parenteral” administration of an immunogenic composition includes, e.g., subcutaneous (s.c.), intravenous (i.v.), intramuscular (i.m.), or intradermal (i.d.) injection, or infusion techniques.

In the context of the field of medicine, the term “prevent” encompasses any activity which reduces the burden of mortality or morbidity from disease. Prevention can occur at primary, secondary and tertiary prevention levels. While primary prevention avoids the development of a disease, secondary and tertiary levels of prevention encompass activities aimed at preventing the progression of a disease and the emergence of symptoms as well as reducing the negative impact of an already established disease by restoring function and reducing disease-related complications.

A “variant” of a polypeptide according to the present invention may be (i) one in which one or more of the amino acid residues are substituted with a conserved or non-conserved amino acid residue (preferably a conserved amino acid residue) and such substituted amino acid residue may or may not be one encoded by the genetic code, (ii) one in which there are one or more modified amino acid residues, e.g., residues that are modified by the attachment of substituent groups, (iii) one in which the polypeptide is an alternative splice variant of the polypeptide of the present invention, (iv) fragments of the polypeptides and/or (v) one in which the polypeptide is fused with another polypeptide, such as a leader or secretory sequence or a sequence which is employed for purification (for example, His-tag) or for detection (for example, Sv5 epitope tag). The fragments include polypeptides generated via proteolytic cleavage (including multi-site proteolysis) of an original sequence. Variants may be post-translationally, or chemically modified. Such variants are deemed to be within the scope of those skilled in the art from the teaching herein. As used herein, the term “variant” includes peptides with at least about 95% identity to the peptides disclosed herein.

Within the meaning of the present invention, the term “conjoint administration” is used to refer to administration of a composition according to the invention and another therapeutic agent simultaneously in one composition, or simultaneously in different compositions, or sequentially (preferably, within a 24 hour period).

In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, New York (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985); Transcription and Translation (B. D. Hames & S. J. Higgins, eds. (1984); Animal Cell Culture (R. I. Freshney, ed. (1986); Immobilized Cells and Enzymes (IRL Press, (1986); B. Perbal, A Practical Guide To Molecular Cloning (1984); F. M. Ausubel et al. (eds.), Current Protocols in Molecular Biology, John Wiley & Sons, Inc. (1994); among others.

### Peptide Compositions of the Invention

Modifications of the amino acid structure of CP1 has led to the discovery of additional peptides that are able to regulate complement activation, such as C1q activity. It was previously demonstrated that substitution of isoleucine with sarcosine at position 8 (IALILEP(Sar)CCQERAA, SEQ ID NO: 4, PA-I8Sar) and position 9 (IALILEPI(Sar)CQERAA, SEQ ID NO: 5, PA-C9Sar) resulted in peptides with increased solubility without PEGylation and enhanced inhibition of biological activity compared to the parent molecule (IALILEPICCQERAA-dPEG24; SEQ ID NO: 3, PA-dPEG24) in in vitro assays of classical complement pathway activation/inhibition, myeloperoxidase (MPO) inhibition, oxidant and NET activity. To determine if more potent peptides could be identified, amino acid variants based on the PA-I8Sar and PA-C9Sar backbone were synthesized and consisted of stapled peptides or peptides with D-amino acids individually substituted at each position in the PA-I8Sar and PA-C9Sar peptide sequence (Table 1). One peptide based on the PA-I8Sar backbone contained a combination of a staple and D-amino acid combinations. Without wishing to be bound by theory, stapling technology can increase peptide stability and enhance biological activity by locking the peptide molecule into a bioactive α-helix secondary structure. Without wishing to be bound by theory, D-amino acid substitutions may impart additional stability to peptides, increasing their in vivo half-life. All but one of these peptides were readily soluble in water and were evaluated for biological activity in the various in vitro assays.

The term “peptide(s),” as used herein, refers to amino acid sequences, which may be naturally occurring, or peptide mimetics, peptide analogs and/or synthetic derivatives (such as for example and not limitation, stapled peptides, sarcosine substitutions, D-amino acid substitutions, and PEGylated peptides) of about 15 amino acids based on SEQ ID NO: 4 or SEQ ID NO: 5. In addition, the peptide may be less than about 15 amino acid residues, such as between about 10 and about 15 amino acid residues and such as peptides between about 5 to about 10 amino acid residues. Peptide residues of, for example, 5, 6, 7, 8, 9, 10, 11, 12, 13, 14, and 15 amino acids are equally likely to be peptides within the context of the present invention. Peptides can also be more than 15 amino acids, such as, for example, 16, 17, 18, 19, and 20, or more amino acids.

The disclosed peptides are generally constrained (that is, have some element of structure as, for example, the presence of amino acids that initiate a 13 turn or (3 pleated sheet, or, for example, are cyclized by the presence of disulfide bonded Cys residues) or unconstrained (that is, linear) amino acid sequences of about 15 amino acid residues, or less than about 15 amino acid residues.

Substitutes for an amino acid within the peptide sequence may be selected from other members of the class to which the amino acid belongs. For example, the nonpolar (hydrophobic) amino acids include alanine, leucine, isoleucine, valine, proline, phenylalanine, tryptophan, and methionine. Amino acids containing aromatic ring structures include phenylalanine, tryptophan, and tyrosine. The polar neutral amino acids include glycine, serine, threonine, cysteine, tyrosine, asparagine, and glutamine. The positively charged (basic) amino acids include arginine and lysine. The negatively charged (acidic) amino acids include aspartic acid and glutamic acid. For example, one or more amino acid residues within the sequence can be substituted by another amino acid of a similar polarity, which acts as a functional equivalent, resulting in a silent alteration.

A conservative change generally leads to less change in the structure and function of the resulting protein. A non-conservative change is more likely to alter the structure, activity, or function of the resulting protein. For example, the peptide of the present disclosure comprises one or more of the following conservative amino acid substitutions: replacement of an aliphatic amino acid, such as alanine, valine, leucine, and isoleucine, with another aliphatic amino acid; replacement of a serine with a threonine; replacement of a threonine with a serine; replacement of an acidic residue, such as aspartic acid and glutamic acid, with another acidic residue; replacement of a residue bearing an amide group, such as asparagine and glutamine, with another residue bearing an amide group; exchange of a basic residue, such as lysine and arginine, with another basic residue; and replacement of an aromatic residue, such as phenylalanine and tyrosine, with another aromatic residue.

Particularly preferred amino acid substitutions include:
a) Ala for Glu or vice versa, such that a negative charge may be reduced;
b) Lys for Arg or vice versa, such that a positive charge may be maintained;
c) Ala for Arg or vice versa, such that a positive charge may be reduced;
d) Glu for Asp or vice versa, such that a negative charge may be maintained;
e) Ser for Thr or vice versa, such that a free —OH can be maintained;
f) Gln for Asn or vice versa, such that a free NH2 can be maintained;
g) Ile for Leu or for Val or vice versa, as roughly equivalent hydrophobic amino acids;
h) Phe for Tyr or vice versa, as roughly equivalent aromatic amino acids; and
i) Ala for Cys or vice versa, such that disulfide bonding is affected.

Substitutes for an amino acid within the peptide sequence may be selected from any amino acids, including, but not limited to alanine, arginine, asparagine, aspartic acid, cysteine, glutamic acid, glutamine, glycine, histidine, isoleucine, leucine, lysine, methionine, phenylalanine, proline, pyrolysine, selenocysteine, serine, threonine, tryptophan, tyrosine, valine, N-formyl-L-methionine, sarcosine, or other N-methylated amino acids. In some embodiments, sarcosine substitutes for an amino acid within the peptide sequence.

Stapling of the peptides can be achieved by using non-native amino acids in desired positions in the peptide that have side chains that can be linked, e.g., by covalent bonding, in order to introduce alpha-helices into the peptide structure. See, e.g., Ali et al., Stapled Peptides Inhibitors: A New Window for Target Drug Discovery, Comput Struct Biotechnol J. 2019; 17: 263-281 and Walensky et al., Hydrocarbon-Stapled Peptides: Principles, Practice, and Progress, J Med Chem. 2014 Aug. 14; 57(15): 6275-6288.

In one embodiment, the invention discloses synthetic peptides derived from human astrovirus coat protein, the peptides comprising the amino acid sequences and modifications of SEQ ID NOs: 6-13. In some embodiments, the invention discloses synthetic peptides derived from human astrovirus coat protein, the peptides comprising the amino acid sequences and modifications of SEQ ID NOs: 6-13 as shown in Table 1 below. The stapled amino acids are indicated by underlining and the D-enantiomeric amino acids are indicated in bold.

TABLE 1
List of Peptides of the Invention.

| SEQ | | |
|---|---|---|
| ID | | |
| NO. | Sequence | Description |
| 1 | PAICQRATATLGTVGSNTSGTTEIEACILL | Astrovirus CP protein |
| 2 | IALILEPICCQERAA | PA (PIC1) |
| 3 | IALILEPICCQERAA-PEG24 | PA-dPEG24; RLS-0071 |
| 4 | IALILEP(Sar)CCQERAA | PA-I8Sar; RLS-0088 |
| 5 | IALILEPI(Sar)CQERAA | PA-C9Sar; RLS-0089 |
| 6 | IAdLILEP(Sar)CCQERAA | PA-0122 (RLS-0122), D-enantiomer |
| | | substitution of SEQ ID NO: 4 |
| 7 | IAR8ILEP(Sar)CS5QERAA | PA-0134 (RLS-0134), stapled |
| | | modification of SEQ ID NO: 4 |
| 8 | IAdLILEP(Sar)CS5QERS5A | PA-0142 (RLS-0142), stapled and D- |
| | | enantiomeric modification of SEQ ID NO: |
| | | 4 |
| 9 | IALILER8I(Sar)CQERS5A | PA-0150 (RLS-0150), stapled |
| | | modification of SEQ ID NO: 5 |
| 10 | IS5LILS5PI(Sar)CQERAA | PA-0152 (RLS-0152), stapled |
| | | modification of SEQ ID NO: 5 |
| 11 | IALILES5I(Sar)CS5ERAA | PA-0154 (RLS-0154), stapled |
| | | modification of SEQ ID NO: 5 |
| 12 | IALILEPI(Sar)dCQERAA | PA-0164 (RLS-0164), D-enantiomeric |
| | | modification of SEQ ID NO: 5 |
| 13 | IALILEPI(Sar)CQERdAA | PA-0168 (RLS-0168, D-enantiomeric |
| | | modification of SEQ ID NO: 5 |

In some embodiments, the peptide sequence has at least about 95%, at least about 96%, at least about 97%, at least about 98%, or at least about 99% sequence identity to SEQ ID NOs: 6-13. In some embodiments, the peptide sequence has at least about 95%, at least about 96%, at least about 97%, at least about 98%, or at least about 99% sequence identity to SEQ ID NOs: 6-8. In some embodiments, the peptide sequence has at least about 95%, at least about 96%, at least about 97%, at least about 98%, or at least about 99% sequence identity to SEQ ID NOs: 9-13.

In one aspect, the invention is a synthetic peptide comprising the amino acid sequence and modifications of SEQ ID NO: 6-13. In some embodiments, the invention is a synthetic peptide comprising the amino acid sequence and modifications of SEQ ID NO: 6-8. In some embodiments, the invention is a synthetic peptide comprising the amino acid sequence and modifications of SEQ ID NO: 9-13.

In another aspect, the invention is a pharmaceutical composition comprising a therapeutically effective amount of at least one synthetic peptide selected from the group consisting of SEQ ID NO: 6-13, and variants thereof, and at least one pharmaceutically acceptable carrier, diluent, or excipient. In another aspect, the invention is a pharmaceutical composition comprising a therapeutically effective amount of at least one synthetic peptide selected from the group consisting of SEQ ID NO: 6-8, and variants thereof, and at least one pharmaceutically acceptable carrier, diluent, or excipient. In another aspect, the invention is a pharmaceutical composition comprising a therapeutically effective amount of at least one synthetic peptide selected from the group consisting of SEQ ID NO: 9-13, and variants thereof, and at least one pharmaceutically acceptable carrier, diluent, or excipient. In another aspect, the pharmaceutical composition further comprises another D-enantiomeric and/or stapled peptide form of SEQ ID NO: 4 and/or SEQ ID NO: 5. In another aspect, the pharmaceutical composition further comprises one or more of SEQ ID NO: 2, 3, 4, and/or 5, and variants thereof.

The disclosed peptides can selectively regulate C1q and MBL activation without affecting alternative pathway activity and are, thus, ideal for preventing and treating diseases mediated by the dysregulated activation of the classical and lectin pathways, respectively. Specific blockade of classical and lectin pathways are particularly needed, as both of these pathways have been implicated in ischemia-reperfusion induced injury in many animal models. [Castellano et al., “Therapeutic targeting of classical and lectin pathways of complement protects from ischemia-reperfusion-induced renal damage.” Am J Pathol. 2010; 176(4):1648-59; Lee et al., “Early complement factors in the local tissue immunocomplex generated during intestinal ischemia/reperfusion injury.” Mol. Immunol. 2010 February; 47(5): 972-81; Tjernberg, et al., “Acute antibody-mediated complement activation mediates lysis of pancreatic islets cells and may cause tissue loss in clinical islet transplantation.” Transplantation. 2008 Apr. 27; 85(8):1193-9; Zhang et al. “The role of natural IgM in myocardial ischemia-reperfusion injury.” J Mol Cell Cardiol. 2006 July; 41(1):62-7). The alternative pathway is essential for immune surveillance against invading pathogens, and humans with alternative pathway defects suffer severe bacterial infections. By binding and inactivating C1q and MBL, the peptides can efficiently regulate classical and lectin pathway activation while leaving the alternative pathway intact.

The term “regulate,” as used herein, refers to i) controlling, reducing, inhibiting or regulating the biological function of an enzyme, protein, peptide, factor, byproduct, or derivative thereof, either individually or in complexes; ii) reducing the quantity of a biological protein, peptide, or derivative thereof, either in vivo or in vitro; or iii) interrupting a biological chain of events, cascade, or pathway known to comprise a related series of biological or chemical reactions. The term “regulate” may thus be used, for example, to describe reducing the quantity of a single component of the complement cascade compared to a control sample, reducing the rate or total amount of formation of a component or complex of components, or reducing the overall activity of a complex process or series of biological reactions, leading to such outcomes as cell lysis, formation of convertase enzymes, formation of complement-derived membrane attack complexes, inflammation, or inflammatory disease. In an in vitro assay, the term “regulate” may refer to the measurable change or reduction of some biological or chemical event, but the person of ordinary skill in the art will appreciate that the measurable change or reduction need not be total to be “regulatory.”

In some embodiments, the present invention relates to therapeutically active peptides having the effects of regulating the complement system.

Modulation of C1q Interaction with C1q Receptors

C1q interactions with C1q receptors appear to play important roles in homeostatic functions such as scavenging of apoptotic cellular debris and immune complexes as well as T cell signaling through antigen presenting cells (macrophage and dendritic cells). Currently, no clinical pharmacological agents modulate the interaction of C1q with C1q receptors.

The disclosed peptides can be used to block C1q binding to C1q receptors, including calreticulin/cC1qR. The ability of the disclosed peptides to block binding of C1q to cellular receptors may have an important role in modulating intracellular signaling processes mediated by C1q binding to C1q receptors.

Myeloperoxidase (MPO) Activity

Myeloperoxidase (MPO) is an enzyme from neutrophils that creates hypochlorite (bleach) in acute inflammation and damages invading and host cells alike. This enzyme is known to be destructive to host tissues in many diseases.

In some embodiments, the peptides disclosed herein blocked the enzymatic activity of MPO. In some embodiments, MPO activity present in the lysates of purified human neutrophils can be directly inhibited by the peptides. In some embodiments, the invention demonstrates that the peptides have anti-inflammatory activity.

Oxidant Activity

Oxidant activity resulting in the formation of reactive oxygen species can be formed in acute inflammation leading to host cell and tissue damage. In some embodiments, the peptides disclosed herein possess antioxidant activity against oxidant generating molecules such as MPO.

Hemolysis Inhibition

The peptides of the invention, including SEQ ID NO: 6-13, can block complement-mediated lysis of AB human red blood cells (RBC) by 0 serum in vitro. This assay mimics ABO incompatibility.

PD-1/PD-L1 Binding Inhibition

The peptides of this invention, including SEQ ID NO: 6-13, can block PD-1 binding to PD-L1 in vitro. This assay mimics the binding between PD-1 on T cells and its ligand PD-L1 on the surface of cancer cells.

### Pharmaceutical Compositions of the Invention

The present disclosure provides pharmaceutical compositions capable of regulating the complement system, comprising at least one peptide, as discussed above, and at least one pharmaceutically acceptable carrier, diluent, stabilizer, or excipient. Pharmaceutically acceptable carriers, excipients, or stabilizers are nontoxic to recipients at the dosages and concentrations employed. They can be solid, semi-solid, or liquid. The pharmaceutical compositions of the present invention can be in the form of tablets, pills, powders, lozenges, sachets, cachets, elixirs, suspensions, emulsions, solutions, or syrups.

The pharmaceutical compositions of the present invention are prepared by mixing the peptide(s) having the appropriate degree of purity with pharmaceutically acceptable carriers, diluents, or excipients. Examples of formulations and methods for preparing such formulations are well known in the art. The pharmaceutical compositions of the present invention are useful as a prophylactic and therapeutic agent for various disorders and diseases, as set forth above. In one embodiment, the composition comprises a therapeutically effective amount of at least one peptide. In another embodiment, the composition comprises at least one other active ingredient effective in regulating the complement system. In another embodiment, the composition comprises at least one other active ingredient effective in treating at least one disease associated with the complement system. In another embodiment, the composition comprises at least one other active ingredient effective in treating at least one disease that is not associated with the complement system. The term “therapeutically effective amount,” as used herein, refers to the total amount of each active component that is sufficient to show a benefit to the subject.

The therapeutically effective amount of the peptide(s) varies depending on several factors, such as the condition being treated, the severity of the condition, the time of administration, the route of administration, the rate of excretion of the peptide(s) employed, the duration of treatment, the co-therapy involved, and the age, gender, weight, and condition of the subject, etc. One of ordinary skill in the art can determine the therapeutically effective amount. Accordingly, one of ordinary skill in the art may need to titer the dosage and modify the route of administration to obtain the maximal therapeutic effect.

The effective daily dose generally is within the range of from about 0.001 to about 200 milligrams per kilogram (mg/kg) of body weight, including about 5 to about 160 mg/kg, about 10 to about 160 mg/kg, about 40 mg/kg to about 160 mg/kg, and about 40 mg/kg to about 100 mg/kg. This dose can be achieved through a 1-6 time(s) daily dosing regimen. Alternatively, optimal treatment can be achieved through a sustained release formulation with a less frequent dosing regimen.

In another aspect, the invention is a pharmaceutical composition comprising a therapeutically effective amount of at least one synthetic peptide selected from the group consisting of SEQ ID NO: 6-13, and variants thereof, and at least one pharmaceutically acceptable carrier, diluent, or excipient. In another aspect, the invention is a pharmaceutical composition comprising a therapeutically effective amount of at least one synthetic peptide selected from the group consisting of SEQ ID NO: 6-8, and variants thereof, and at least one pharmaceutically acceptable carrier, diluent, or excipient. In another aspect, the invention is a pharmaceutical composition comprising a therapeutically effective amount of at least one synthetic peptide selected from the group consisting of SEQ ID NO: 9-13, and variants thereof, and at least one pharmaceutically acceptable carrier, diluent, or excipient. In another aspect, the pharmaceutical composition further comprises another D-enantiomeric and/or stapled peptide form of SEQ ID NO: 4 and/or SEQ ID NO: 5, and at least one pharmaceutically acceptable carrier, diluent, or excipient. In another aspect, the pharmaceutical composition further comprises one or more of SEQ ID NO: 2, 3, 4, and/or 5, and variants thereof, and at least one pharmaceutically acceptable carrier, diluent, or excipient.

The compositions of the invention can comprise a carrier and/or excipient. While it is possible to use a peptide of the present invention for therapy as is, it may be preferable to administer it in a pharmaceutical formulation, e.g., in admixture with a suitable pharmaceutical excipient and/or carrier selected with regard to the intended route of administration and standard pharmaceutical practice. The excipient and/or carrier must be “acceptable” in the sense of being compatible with the other ingredients of the formulation and not deleterious to the recipient thereof. Acceptable excipients and carriers for therapeutic use are well known in the pharmaceutical art, and are described, for example, in Remington: The Science and Practice of Pharmacy. Lippincott Williams & Wilkins (A. R. Gennaro edit. 2005). The choice of pharmaceutical excipient and carrier can be selected with regard to the intended route of administration and standard pharmaceutical practice. Oral formulations readily accommodate additional mixtures, such as, e.g., milk, yogurt, and infant formula. Solid dosage forms for oral administration can also be used and can include, e.g., capsules, tablets, caplets, pills, troches, lozenges, powders, and granules. Non-limiting examples of suitable excipients include, e.g., diluents, buffering agents (e.g., sodium bicarbonate), preservatives, stabilizers, binders, compaction agents, lubricants, dispersion enhancers, disintegration agents, antioxidants, flavoring agents, sweeteners, and coloring agents. Those of relevant skill in the art are well able to prepare suitable solutions.

In one embodiment of any of the compositions of the invention, the composition is formulated for delivery by a route such as, e.g., oral, topical, rectal, mucosal, sublingual, nasal, naso/oro-gastric gavage, parenteral, intraperitoneal, intradermal, transdermal, intrathecal, nasal, and intracheal administration. In one embodiment of any of the compositions of the invention, the composition is in a form of a liquid, foam, cream, spray, powder, or gel. In one embodiment of any of the compositions of the invention, the composition comprises a buffering agent (e.g., sodium bicarbonate).

Administration of the compounds and compositions in the methods of the invention can be accomplished by any method known in the art. Non-limiting examples of useful routes of delivery include oral, rectal, fecal (by enema), and via naso/oro-gastric gavage, as well as parenteral, intraperitoneal, intradermal, transdermal, intrathecal, nasal, and intracheal administration. The active agent may be systemic after administration or may be localized by the use of regional administration, intramural administration, or use of an implant that acts to retain the active dose at the site of implantation.

The useful dosages of the compounds and formulations of the invention can vary widely, depending upon the nature of the disease, the patient's medical history, the frequency of administration, the manner of administration, the clearance of the agent from the host, and the like. The initial dose may be larger, followed by smaller maintenance doses. The dose may be administered as infrequently as weekly or biweekly, or fractionated into smaller doses and administered daily, semi-weekly, etc., to maintain an effective dosage level. It is contemplated that a variety of doses may be effective to achieve a therapeutic effect. While it is possible to use a compound of the present invention for therapy as is, it may be preferable to administer it in a pharmaceutical formulation, e.g., in admixture with a suitable pharmaceutical excipient, diluent or carrier selected with regard to the intended route of administration and standard pharmaceutical practice. The excipient, diluent and/or carrier must be “acceptable” in the sense of being compatible with the other ingredients of the formulation and not deleterious to the recipient thereof. Acceptable excipients, diluents, and carriers for therapeutic use are well known in the pharmaceutical art, and are described, for example, in Remington: The Science and Practice of Pharmacy. Lippincott Williams & Wilkins (A. R. Gennaro edit. 2005). The choice of pharmaceutical excipient, diluent, and carrier can be selected with regard to the intended route of administration and standard pharmaceutical practice.

Formulations suitable for parenteral administration include aqueous and nonaqueous, isotonic sterile injection solutions, which can contain antioxidants, buffers, bacteriostats, and solutes that render the formulation isotonic with the blood of the intended recipient, and aqueous and nonaqueous sterile suspensions that can include suspending agents, solubilizers, thickening agents, stabilizers, and preservatives.

Solutions or suspensions can include any of the following components, in any combination: a sterile diluent, including by way of example without limitation, water for injection, saline solution, fixed oil, polyethylene glycol, glycerine, propylene glycol or other synthetic solvent; antimicrobial agents, such as benzyl alcohol and methyl parabens; antioxidants, such as ascorbic acid and sodium bisulfite; chelating agents, such as ethylenediaminetetraacetic acid (EDTA); buffers, such as acetates, citrates and phosphates; and agents for the adjustment of tonicity, such as sodium chloride or dextrose.

In instances in which the agents exhibit insufficient solubility, methods for solubilizing agents may be used. Such methods are known to those of skill in this art, and include, but are not limited to, using co-solvents, such as, e.g., dimethylsulfoxide (DMSO), using surfactants, such as TWEEN®80, or dissolution in aqueous sodium bicarbonate. Pharmaceutically acceptable derivatives of the agents may also be used in formulating effective pharmaceutical compositions.

The composition can contain along with the active agent, for example and without limitation: a diluent such as lactose, sucrose, dicalcium phosphate, or carboxymethylcellulose; a lubricant, such as magnesium stearate, calcium stearate and talc; and a binder such as starch, natural gums, such as gum acacia gelatin, glucose, molasses, polyvinylpyrrolidone, celluloses and derivatives thereof, povidone, crospovidones and other such binders known to those of skill in the art. Liquid pharmaceutically administrable compositions can, for example, be prepared by dissolving, dispersing, or otherwise mixing an active agent as defined above and optional pharmaceutical adjuvants in a carrier, such as, by way of example and without limitation, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents, emulsifying agents, or solubilizing agents, pH buffering agents and the like, such as, by way of example and without limitation, acetate, sodium citrate, cyclodextrin derivatives, sorbitan monolaurate, triethanolamine sodium acetate, triethanolamine oleate, and other such agents. Actual methods of preparing such dosage forms are known, or will be apparent, to those skilled in this art (e.g., Remington's Pharmaceutical Sciences, Mack Publishing Company, Easton, Pa., 15th Edition, 1975). The composition or formulation to be administered will, in any event, contain a quantity of the active agent in an amount sufficient to alleviate the symptoms of the treated subject.

The active agents or pharmaceutically acceptable derivatives may be prepared with carriers that protect the agent against rapid elimination from the body, such as time release formulations or coatings. The compositions may include other active agents to obtain desired combinations of properties.

Parenteral administration, generally characterized by injection, either subcutaneously, intramuscularly or intravenously, is also contemplated herein. Injectables can be prepared in conventional forms, either as liquid solutions or suspensions, solid forms suitable for solution or suspension in liquid prior to injection, or as emulsions. Suitable excipients include, by way of example and without limitation, water, saline, dextrose, glycerol or ethanol. In addition, if desired, the pharmaceutical compositions to be administered may also contain minor amounts of non-toxic auxiliary substances, such as wetting or emulsifying agents, pH buffering agents, stabilizers, solubility enhancers, and other such agents, such as, for example, sodium acetate, sorbitan monolaurate, triethanolamine oleate and cyclodextrins.

Lyophilized powders can be reconstituted for administration as solutions, emulsions, and other mixtures or formulated as solids or gels. The sterile, lyophilized powder is prepared by dissolving an agent provided herein, or a pharmaceutically acceptable derivative thereof, in a suitable solvent. The solvent may contain an excipient which improves the stability or other pharmacological component of the powder or reconstituted solution, prepared from the powder. Excipients that may be used include, but are not limited to, dextrose, sorbital, fructose, corn syrup, xylitol, glycerin, glucose, sucrose or other suitable agent. The solvent may also contain a buffer, such as citrate, sodium or potassium phosphate or other such buffer known to those of skill in the art at, typically, about neutral pH. Subsequent sterile filtration of the solution followed by lyophilization under standard conditions known to those of skill in the art provides the desired formulation. Generally, the resulting solution can be apportioned into vials for lyophilization. Each vial can contain, by way of example and without limitation, a single dosage (10-1000 mg, such as 100-500 mg) or multiple dosages of the agent. The lyophilized powder can be stored under appropriate conditions, such as at about 4° C. to room temperature. Reconstitution of this lyophilized powder with water for injection provides a formulation for use in parenteral administration.

### Methods of Use

Another aspect of the invention provides a method of regulating the complement system comprising administering a therapeutically effective amount of the peptides and/or the pharmaceutical compositions of the invention to a subject in need thereof. Activation of the classical complement pathway has been shown to contribute to tumor progression for certain cancers like clear cell renal cell carcinoma such that inhibition of the classical complement pathway may be a therapeutic approach to slowing the progression of such tumors.

In another aspect, the invention provides a method of inhibiting myeloperoxidase activity comprising administering a therapeutically effective amount of the peptides and/or the pharmaceutical compositions of the invention to a subject in need thereof.

In another aspect, the invention provides a method of inhibiting oxidant activity comprising administering a therapeutically effective amount of the peptides and/or the pharmaceutical compositions of the invention to a subject in need thereof.

In another aspect, the invention provides a method of inhibiting PD-L1 activity comprising administering a therapeutically effective amount of the peptides and/or the pharmaceutical compositions of the invention to a subject in need thereof.

### Combination Therapies

A further embodiment of the invention provides a method of regulating the complement system, comprising administering to a subject a pharmaceutical composition of the present invention. While the pharmaceutical compositions of the present invention can be administered as the sole active pharmaceutical agent, they can also be used in combination with one or more therapeutic or prophylactic agent(s) that is(are) effective for regulating the complement system. In this aspect, the method of the present invention comprises administrating a pharmaceutical composition of the present invention before, concurrently, and/or after one or more additional therapeutic or prophylactic agents effective in regulating the complement system.

The pharmaceutical compositions of the present invention can be administered with additional agent(s) in combination therapy, either jointly or separately, or by combining the pharmaceutical compositions and the additional agent(s) into one composition. The dosage is administered and adjusted to achieve maximal regulation of the complement system. For example, both the pharmaceutical compositions and the additional agent(s) are usually present at dosage levels of between about 10% and about 150%, more preferably, between about 10% and about 80%, of the dosage normally administered in a mono-therapy regimen.

### Examples

The present invention is also described and demonstrated by way of the following examples. However, the use of these and other examples anywhere in the specification is illustrative only and in no way limits the scope and meaning of the invention or of any exemplified term. Likewise, the invention is not limited to any particular preferred embodiments described here. Indeed, many modifications and variations of the invention may be apparent to those skilled in the art upon reading this specification, and such variations can be made without departing from the invention in spirit or in scope. The invention is therefore to be limited only by the terms of the appended claims along with the full scope of equivalents to which those claims are entitled.

### Example 1: Characterization of SEQ ID NOs: 6-13

The inventors found that a sarcosine amino acid substitution scan of PA-dPEG24 revealed that substitution of this amino acid at different positions of the peptide, resulted in peptides that were soluble in water in the absence of PEGylation and also displayed increased inhibitory activity in in vitro assays of classical complement pathway activation, MPO activation, NET formation and antioxidant activity [11]. Two variants in which sarcosine replaced isoleucine at position 8 (PA-I8Sar) or cysteine at position 9 (PA-C9Sar) displayed increased inhibitory activity in the respective assays. Using the peptide backbone of both PA-I8Sar and PA-C9Sar, the inventors created peptide peptides with D-amino acid substitutions and/or engineered stapling that display increased potency in the various functional assays and retain aqueous solubility in the absence of PEGylation.

### Materials and Reagents

Peptides were synthesized by New England Peptide (Gardner, MA) to >90% purity (Table 1). Stapled peptides were produced through a one-component stapling technique employing S-pentenylalanine (S5) at i,i+4 positions for one-turn stapling or combining either R-octenylalanine (R8)/S-pentenylalanine (S5) at i,i+7 positions. D-enantiomer forms of each amino acid individually substituted at various position in the PA-I8Sar and PA-C9Sar peptide sequence except for sarcosine at position 8 (PA-I8Sar) and position 9 (PA-C9Sar) as no enantiomeric form exists. All peptides were dissolved in water and the pH was adjusted with NaOH. Purified C1q was purchased from Complement Technology (Tyler, TX). Purified MPO was purchased from Lee BioSolutions (Maryland Heights, MO) and tetramethylbenzidine (TMB) was purchased from Thermo Fisher (Waltham MA). Buffers included Complement permissive GVBS⁺⁺ buffer (veronal-buffered saline with 0.1% gelatin, 0.15 mM CaCl₂, and 1 mM MgCl₂ [12]).

### Methods

### Normal Human Serum (NHS)

Blood type O normal human serum (NHS) was prepared as previously described [12]. Briefly, blood from at least 4 healthy human donors was collected in Vacutainer tubes without additives (red top). The blood was incubated for 30 minutes at room temperature and 2 hours on ice to clot and the serum separated. The sera was then pooled, aliquoted and frozen at −80° C.

### Hemolytic Assays of Complement Activity

For hemolytic complement assays, human red blood cells (RBCs) from type AB donors were purified, washed, and standardized to 1.0×10⁹ cells/ml, as previously described [13]. Human sera from type 0 donors at a 15% final concentration was combined with 0.5 mM of the peptides and the volume was brought up to 0.2 ml with GVBS⁺⁺ and 5.0×10⁷ RBCs. The samples were incubated for 1 hour at 37° C. and then spun at 3,000 rpm for 5 minutes and the supernatant was collected and read at 412 nm. Values are represented as a percent of the positive control, which consists of human 0 sera and AB red blood cells in GVBS⁺⁺ buffer.

### C1q Binding Assay

The C1q binding assay was performed as previously described [11]. Briefly, an Immunlon-2 HB ELISA plate was coated with 1 μg/ml C1q in bicarbonate buffer overnight at 4° C. The plates were washed with PBS-T (phosphate buffered saline+0.1% Tween) and then blocked with 1% gelatin/PBS for 2 hours at room temperature. After washing, the plates were incubated with the peptides starting at 2.5 mg/ml and then serially diluted in 1% gelatin/PBS for 1 hour at room temperature followed by washing. Plates were then probed with rabbit antibody raised against the lead peptide IALILEPICCQERAA (SEQ ID NO: 2) that lacked PEGylation [11] at 1:1000 in 1% gelatin/PBS for 1 hour at room temperature followed by a goat anti-rabbit HRP (Sigma Aldrich, St Louis, MO) at 1:1,000 in 1% gelatin/PBS for 1 hour at room temperature with a washing step in between. After addition of TMB substrate solution to the wells, the reaction was stopped using 1N H₂SO₄, and the plate read on a BioTek Synergy HT plate reader at 450 nm.

### MPO Activity Assay

The MPO activity assay was performed as previously described [10]. Briefly, peptides were diluted to 12 mg/ml and serially titrated in a 96 well plate at a volume of 0.02 ml. MPO was diluted to 20 μg/ml and 0.02 ml was added to the titrated peptides. TMB (3,30,5,50-tetramethylbenzidine) (0.1 ml) was added to each well for 2 minutes, followed by 0.1 ml of 2.5 N H₂SO₄ for another 2 minutes, and then read on a 96 well plate reader (BioTek) at 450 nm.

### Total Antioxidant Capacity Assay

As previously reported [6], the TAC (Total Antioxidant Capacity) Assay (Cell Biolabs, Inc, San Diego, CA) was used to measure the antioxidant capacity of the PIC1 variants based on the reduction of copper (II) to copper (I). The kit protocol was performed per the manufacturer's recommendations.

PD-1: PD-L1 Inhibitor Screening ELISA Assay Pair

This inhibitor screening ELISA pair was designed to facilitate the identification and characterization of new PD-1 pathway inhibitors. In this assay, biotinylated human PD-1 was bound to immobilized human PD-L1 and a colorimetric sandwich ELISA platform was used. Plates were coated with human PD-L1 followed by incubation with various PIC1 peptides. Human PD-1-Biotin was then allowed to bind the coated human PD-L1 followed by addition of streptavidin-HRP. TMB was then added as the colorimetric HRP substrate and the wells of the plate analyzed in a plate reader at an absorbance of 450 nm.

### Statistical Analysis

Quantitative data were analyzed determining means, standard error (SEM), and Student's t-test [14] using Excel (Microsoft, Redmond, WA).

### Results

### Peptides

It was previously demonstrated that substitution of isoleucine with sarcosine at position 8 (IALILEP(Sar)CCQERAA, SEQ ID NO:4, PA-I8Sar) and position 9 (IALILEPI(Sar)CQERAA, SEQ ID NO: 5, PA-C9Sar) resulted in peptides with increased solubility without PEGylation and enhanced inhibition of biological activity compared to the parent molecule (IALILEPICCQERAA-dPEG24, SEQ ID NO:3) in in vitro assays of classical complement pathway, MPO, oxidant and NET activity [11]. To determine if more potent peptides could be identified, amino acid variants based on the PA-I8Sar and PA-C9Sar backbone were synthesized and consisted of stapled peptides or peptides with D-amino acids individually substituted at each position in the PA-I8Sar and PA-C9Sar peptide sequence (Tables 1 and 2, respectively). One peptide based on the PA-I8Sar backbone contained a combination of a staple and D-amino acid combinations (PA-0142). Stapling technology has been demonstrated to increase peptide stability and enhance biological activity by locking the peptide molecule into a bioactive α-helix secondary structure [2] whereas D-amino acid substitutions can impart additional stability to natural peptides increasing their in vivo half-life [15]. Each of these peptides were readily soluble in water and were evaluated for biological activity in the various in vitro assays.

### Complement Inhibition and C1q Binding

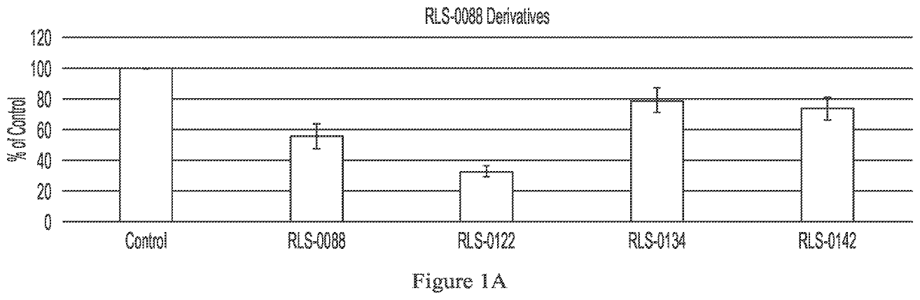

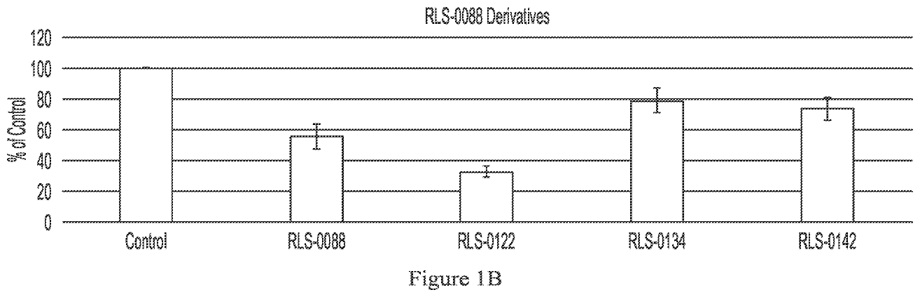

To assess the extent to which the peptide variants inhibit antibody-initiated complement activation, an ABO incompatibility ex vivo assay was utilized in which purified erythrocytes from a ‘type AB+’ donor are incubated with sera from a ‘type 0’ subject containing anti-A and anti-B antibodies [13]. Peptides were tested at a concentration of 0.8 mg/ml (˜0.5 mM). For peptides based on the PA-I8Sar backbone, peptides PA-0122, PA-0134 and PA-0142 which consisted of D-amino acid substitutions (PA-0122), a stapled peptides (PA-0134) and a stapled and D-amino acid substitutions (PA-0142) inhibited ABO incompatible hemolysis to a similar (PA-0134 and PA-0142) or greater extent compared (PA-0122) to the PA-I8Sar control (FIG. 1A).

Stapled and D-amino acid variants of the PA-C9Sar molecule were next tested for inhibition of complement in the hemolytic assay. In contrast to the PA-I8Sar variants, all of the peptides based on the PA-C9Sar molecule inhibited complement activity more than PA-C9Sar (FIG. 1B).

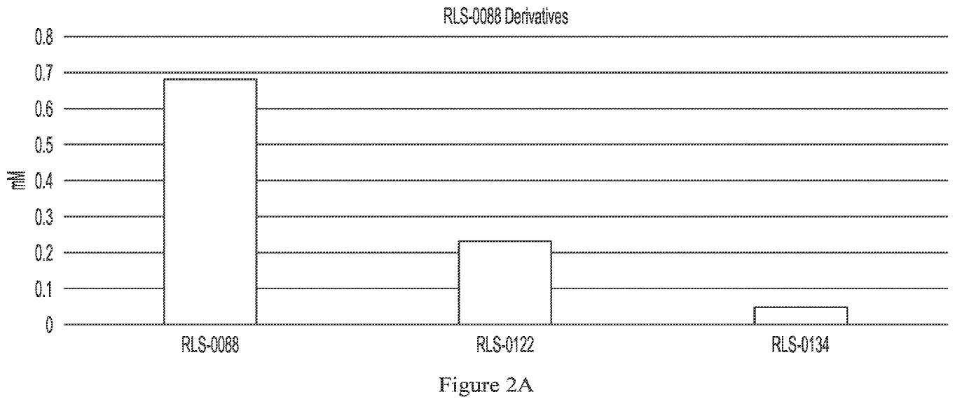

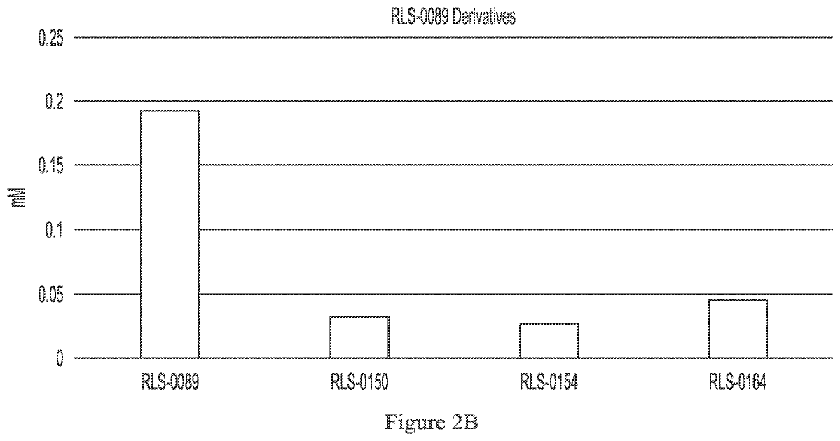

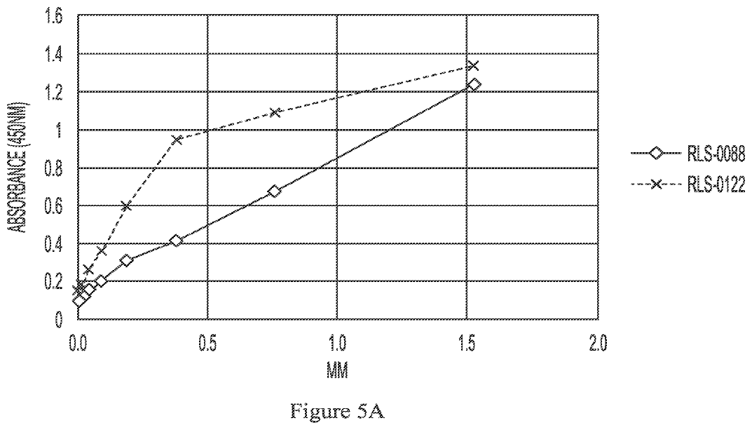

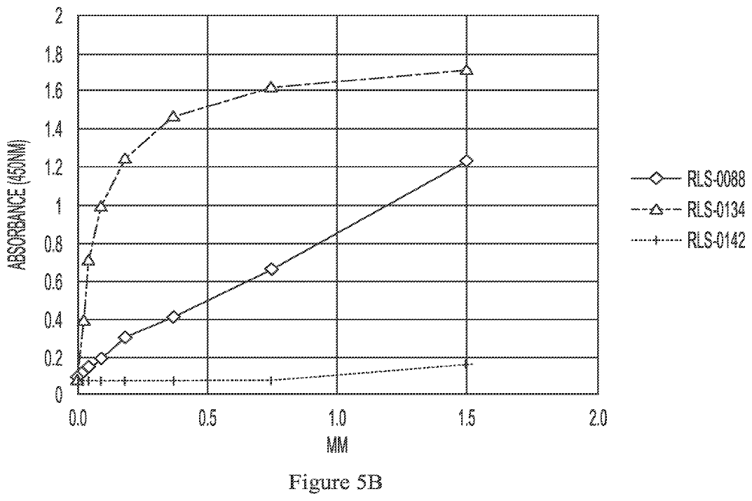

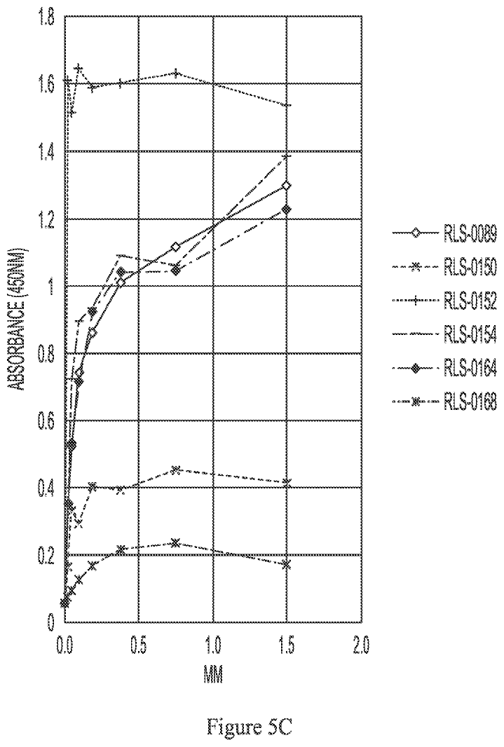

The astrovirus capsid protein and the PIC1 molecules derived from it inhibit classical complement pathway activation by binding to the pattern recognition molecule C1q [3,10,11]. The inventors next tested peptide variant binding to C1q in an ELISA-type assay in which C1q is used as the capture substrate and bound peptide detected with a rabbit polyclonal antibody against the peptide portion of PA-dPEG24 (IALILEPICCQERAA-d24; SEQ ID NO: 3) [11]. Binding curves were derived for the stapled and D-amino acid peptides based on the PA-I8Sar and PA-C9Sar (FIG. 5A-5C) backbone, from which half-maximal binding concentrations were calculated (FIGS. 2A and B, respectively). For the peptides based on the PA-I8Sar backbone, these binding curves and half-maximal binding calculations demonstrate that the D-amino acid peptide PA-0122 and stapled peptide PA-0134 had markedly increased binding to C1q compared with PA-I8Sar. For the peptides based on the PA-C9Sar backbone, C1q was bound to a greater degree than the parent peptide (FIG. 2B). Surprisingly, while a number of peptides showed superior complement inhibition and C1q binding activity such as PA-0150 (compare FIGS. 1B and 2B), the strength of binding to C1q did not strictly correlate with inhibition of classical pathway complement activation of other peptide variants (e.g., PA-0134) (compare FIGS. 1A and 2A) suggesting that complement inhibitory activity may not be wholly dictated by strength of binding to C1q.

### Myeloperoxidase Inhibition

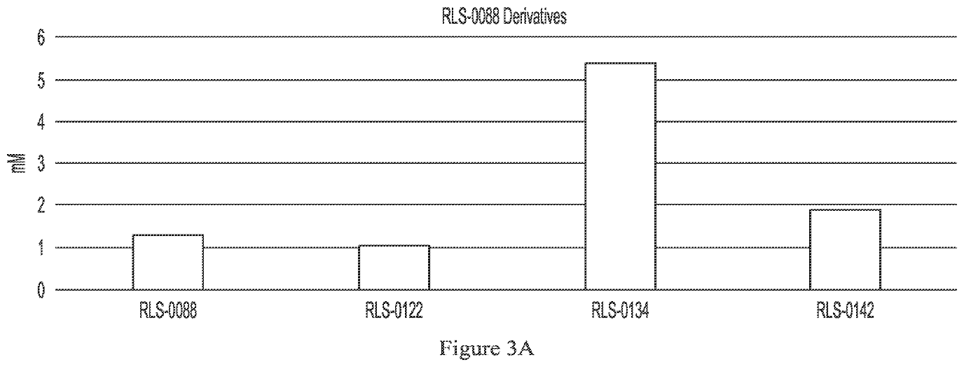

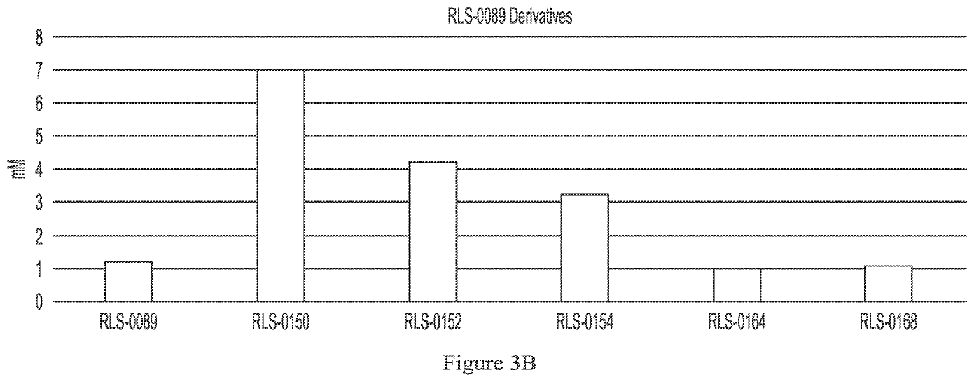

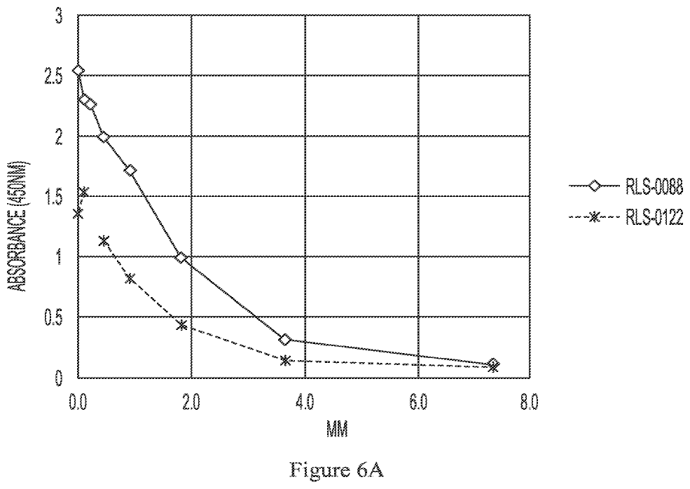

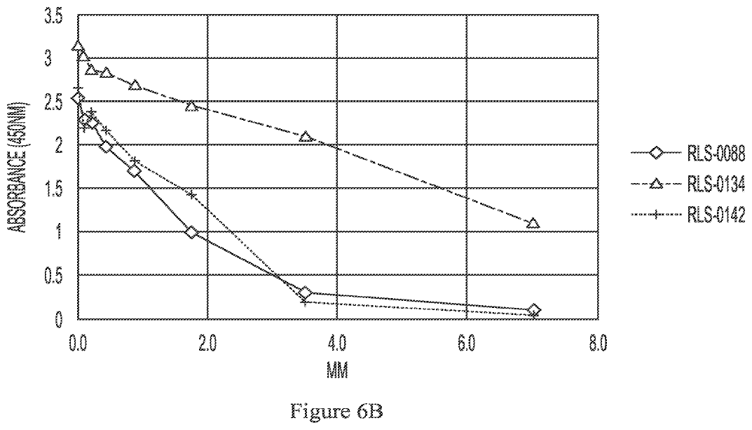

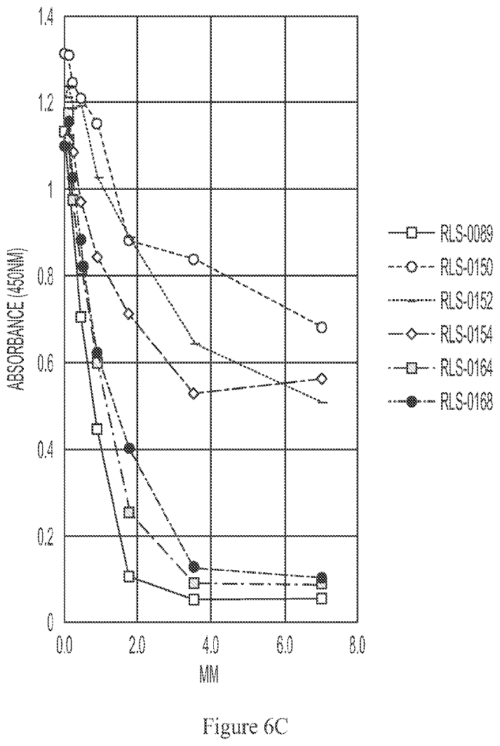

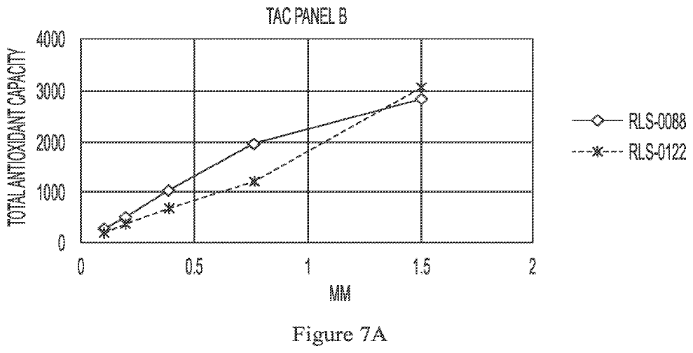

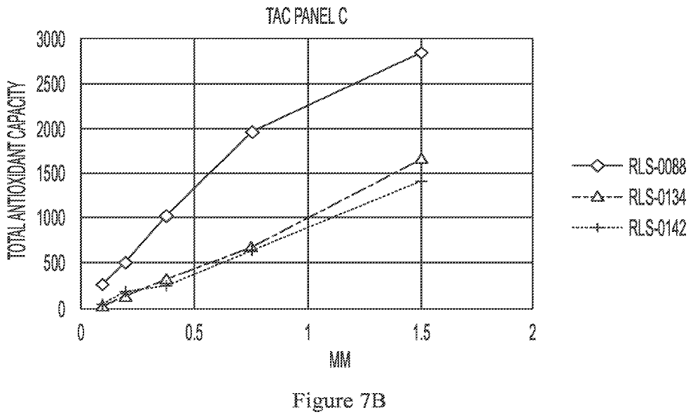

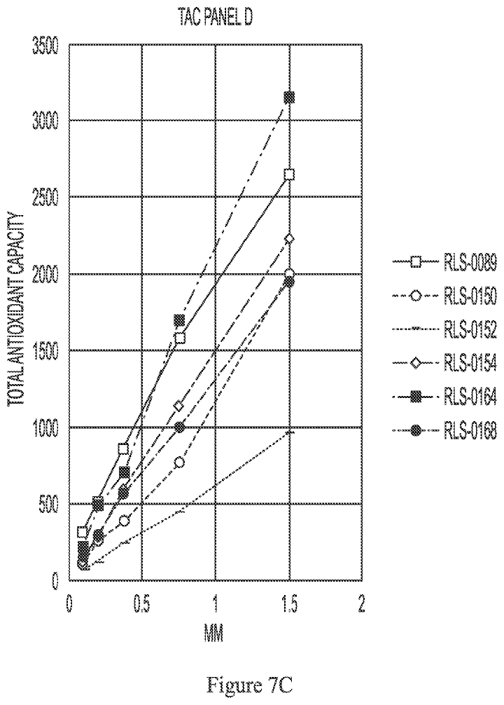

To ascertain the inhibition of MPO activity by the various peptides, a range of concentrations of the stapled peptides and D-amino acid variants were tested (FIG. 3A-3B) and half-maximal activity levels were calculated from the dose-response curves (FIG. 6A-6C, respectively). For the PA-I8Sar peptides, PA-0122 and PA-0142 demonstrated similar levels of MPO inhibition whereas PA-0134 demonstrated reduced inhibition of MPO activity (FIG. 3A). For the PA-C9Sar peptides, the PA-164 and PA0168 maintained similar inhibitory activity as the parent PA-C9Sar peptide whereas PA-0150, PA-0152 and PA-0154 demonstrated decreased MPO inhibition (FIG. 3B). Thus, for both the PA-I8Sar and PA-C9Sar variants, some of the peptides demonstrated varying effects on MPO binding affinity.

### Antioxidant Capacity

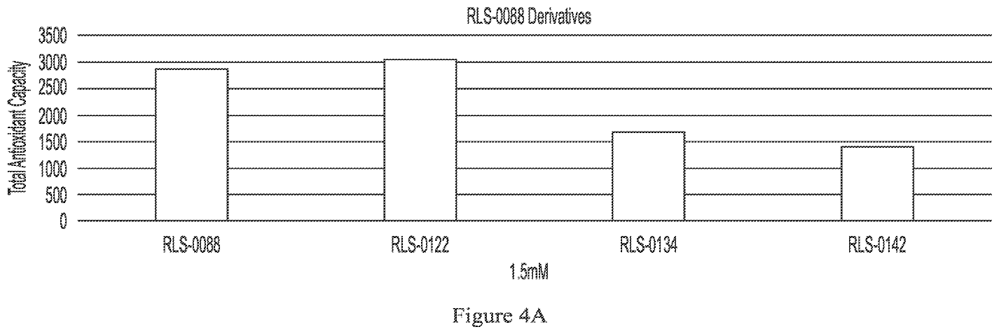

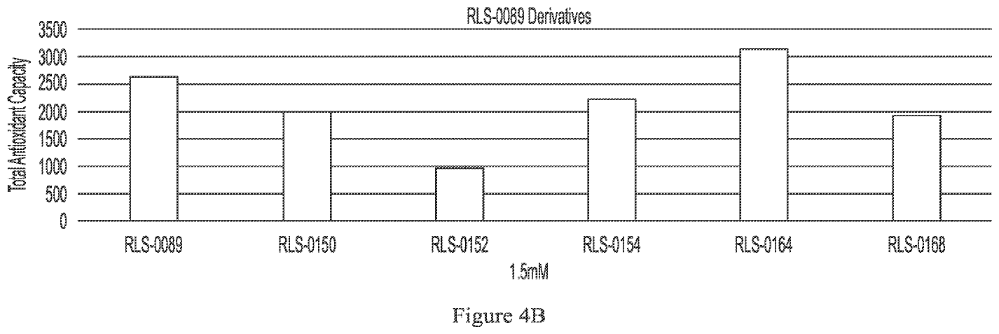

The antioxidant properties for the PIC1 variants were evaluated in a Total Antioxidant Capacity (TAC) assay, as previously reported [6]. Total antioxidant activity over a range of peptide concentrations were determined for the stapled and the D-amino acid peptides based on the PA-I8Sar and PA-C9Sar backbones (FIG. 4A-4B), with the activity of the highest peptide concentration reported (1.5 mM) (FIG. 5A-5C). For the peptides based on the parent PA-I8Sar peptide, PA-0122 showed a slight increase in total antioxidant capacity whereas PA-0134 and PA-0142 had decreased activity compared to the parent peptide. Surprisingly, the majority of peptides based on the PA-C9Sar peptide showed a reduction in total antioxidant capacity with the exception of d-amino acid peptide PA-0164 which had enhanced activity (FIG. 4B). The inventors have previously demonstrated that both vicinal cysteine residues at positions 9 and 10 of the parent PA-dPEG24 peptide (IALILEPICCQERAA-dPEG24, SEQ ID NO: 3) are essential for antioxidant activity with oxidation of both residues inhibiting this function [6]. These data demonstrate that the cysteine at position 10 is sufficient to maintain antioxidant activity and that a D-enantiomer of this cysteine (PA-0164) can enhance this activity.

### Inhibition of PD-1 Binding to PD-L1

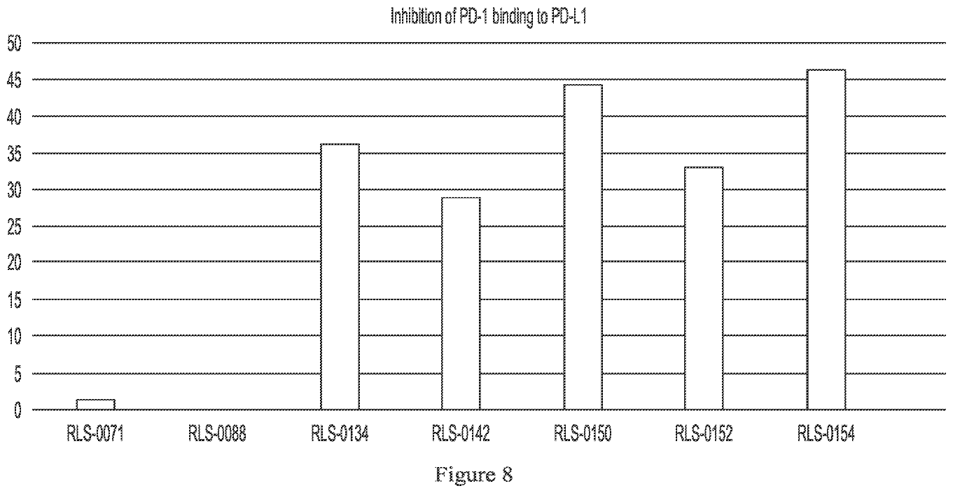

The immune checkpoint pathway is an area of significant interest in cancer research. PD-1 is one of the best characterized checkpoint proteins. The binding between PD-1 and its ligand PD-L1 suppresses T-cell activation and allows cancer cells to escape from body's immune surveillance. Therefore, the pharmaceutical inhibition of PD-1 or its ligand has been considered a promising strategy by many cancer researchers. To ascertain if these PIC1 derivatives could inhibit PD-1 interaction with PD-L1, a commercial ELISA kit was utilized. PA-0071 and PA-0088 did not inhibit the binding of PD-1 to PD-L1. In contrast, PA-0134, PA-0142, PA-0150, PA-0152 and PA-0154 inhibited binding by 29-46% (FIG. 8).

Discussion

The inventors had previously demonstrated that sarcosine substitution of the parental, 15 residue, PEGylated PIC1 molecule (PA-dPEG24) yielded six peptides that were aqueous soluble without PEGylation and had enhanced activity in functional assays of complement, MPO, NETosis and oxidant activity [11]. PA-I8Sar (isoleucine at position 8 substituted for sarcosine) was chosen for further modification by peptide stapling and substitution of D-amino acids to determine if its functional activity in the various assays could be further enhanced. The inventors also performed the same analysis with PA-C9Sar (cysteine at position 9 substituted for sarcosine). While PA-C9Sar did not show as great an enhancement of activity in the various assays compared to PA-I8Sar [11], the inventors were interested to analyze its function in the context of stapling and D-amino acid substitution to see if the single cysteine residue could maintain functional activity. As the inventors have previously demonstrated, both cysteine residues at positions 9 and 10 are critical for the functional activity of the PIC1 molecule [11]. The activity of the variants of PA-I8Sar and PA-C9Sar in the various assays are summarized in Tables 2 and 3.

TABLE 2
PA-18Sar peptides and summary of properties.

| | SEQ | | | | | |
|---|---|---|---|---|---|---|
| | ID | | ABO | C1q | MPO | TAC |
| Name | NO. | Sequence | Hemolysis | Binding | Activity | Activity |
| PA-0088 | 4 | H2N-IALILEP(Sar)CCQERAA- | + | + | + | + |
| | | OH | | | | |
| PA-0122 | 6 | H2N-IAdLILEP(Sar)CCQERAA- | +++ | +++ | ++ | ++ |
| | | OH | | | | |
| PA-0134 | 7 | H2N-IAR8ILEP(Sar)CS5QERAA- | + | ++++ | + | + |
| | | OH | | | | |
| PA-0142 | 8 | H2N-IAdLILEP(Sar)CS5QERS5A- | + | ND | + | + |
| | | OH | | | | |

¹ND: not determined.

TABLE 3
PA-C9Sar peptides and summary of properties.

| | SEQ | | | | | |
|---|---|---|---|---|---|---|
| | ID | | ABO | C1q | MPO | TAC |
| Name | NO. | Sequence | Hemolysis | Binding | Activity | Activity |
| PA-0089 | 5 | H2N-IALILEPI(Sar)CQERAA- | + | + | + | + |
| | | OH | | | | |
| PA-0150 | 9 | H2N-IALILER8I(Sar)CQERS5A- | ++++ | ++++ | + | + |
| | | OH | | | | |
| PA-0152 | 10 | H2N-IS5LILS5PI(Sar)CQERAA- | ++ | ND | + | + |
| | | OH | | | | |
| PA-0154 | 11 | H2N-IALILES5I(Sar)CS5ERAA- | ++ | ++++ | + | + |
| | | OH | | | | |
| PA-0164 | 12 | H2N-IALILEPI(Sar)dCQERAA- | +++ | ++++ | ++ | ++++ |
| | | OH | | | | |
| PA-0168 | 13 | H2N-IALILEPI(Sar)CQERdAA- | +++ | ND | ++ | + |
| | | OH | | | | |

¹ND: not determined. For C1q binding, peptide sequence PA-0168 was not recognized by the polyclonal antibody to the parent peptide sequence, IALILEPICCQERAA-dPEG24 (SEQ ID NO: 3).

The results shown here demonstrate that the functional activities of the PIC1 molecules can be improved significantly with peptide stapling as well as the introduction of non-canonical amino acids. Of interest and surprise was the finding that such modifications can lead to the improvement of one or multiple functional activities of the PIC1 peptide. The ability to isolate peptides with different functional activities can potentially be utilized to target specific inflammatory diseases where dysregulated complement, neutrophil (MPO and NETosis) or oxidant activity plays a predominant role in pathogenesis.

### Example 2. Inhibition of PD-1 Binding to PD-L1

The immune checkpoint pathway is an area of significant interest in cancer research. PD-1 is one of the best characterized checkpoint proteins. The binding between PD-1 and its ligand PD-L1 suppresses T-cell activation and allows cancer cells to escape from body's immune surveillance. Therefore, the pharmaceutical inhibition of PD-1 or its ligand has been considered a promising strategy by many cancer researchers. To ascertain if these PIC1 derivatives could inhibit PD-1 interaction with PD-L1, a commercial ELISA kit was utilized. PA-0071 and PA-0088 did not inhibit the binding of PD-1 to PD-L1. In contrast, PA-0134, PA-0142, PA-0150, PA-0152 and PA-0154 inhibited binding by 29-46% (FIG. 8).

### Example 3. Binding of PD-1, PD-L1 and CTLA-4 by PIC1 Peptides RLS-0134 and RLS-0150

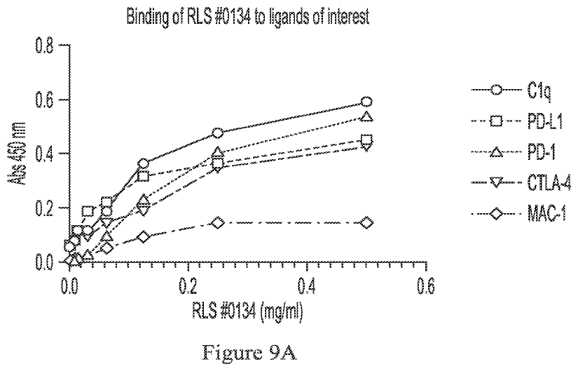

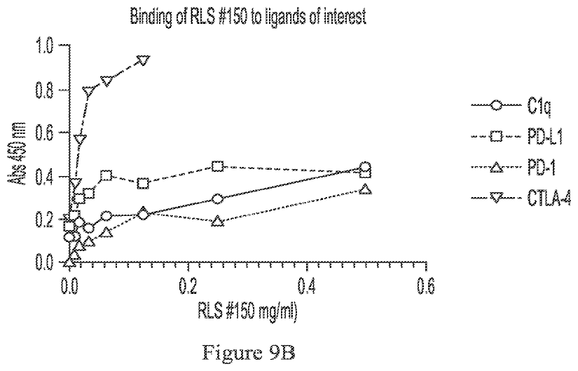

The inventors further evaluated if RLS-0134 and RLS-0150 could bind to the well characterized checkpoint inhibitor, cytotoxic T-lymphocyte associated protein 4 (CTLA-4 aka CD152). As with PD1/PD-L1 interactions, CTLA-4 is a check point protein upregulated on the surface of cancer cells that engages ligands CD80 or CD86 on the surface of T-cells and suppresses T-cell activation allowing the cancer cells to escape destruction by the immune system. Therefore, the pharmaceutical inhibition of CTLA-4 or its ligand has been considered a promising strategy by many cancer researchers and could be a therapeutic target in cancer immunotherapy. To ascertain the ability of select PIC1 peptides to bind CTLA-4, a binding assay was performed in which these proteins were coated on a microtiter plate followed by incubation with increasing amounts of either RLS-0134 and RLS-0150. Plates coated with PD-1, PD-L1 and C1q served as a positive control for peptide binding whereas MAC-1 served as a negative control for peptide binding. RLS-0134 showed dose-dependent binding to PD-1, PD-L1 and C1q as expected and also bound CTLA-4 but showed minimal binding to MAC-1 (FIG. 9A). RLS-0150 also demonstrated dose-dependent binding to PD-1, PD-L1 and C1q with much stronger binding to CTLA-4 (FIG. 9B).

### Example 4. Activity of PIC1 Peptides in a CTLA-4 Blockade Bioassay

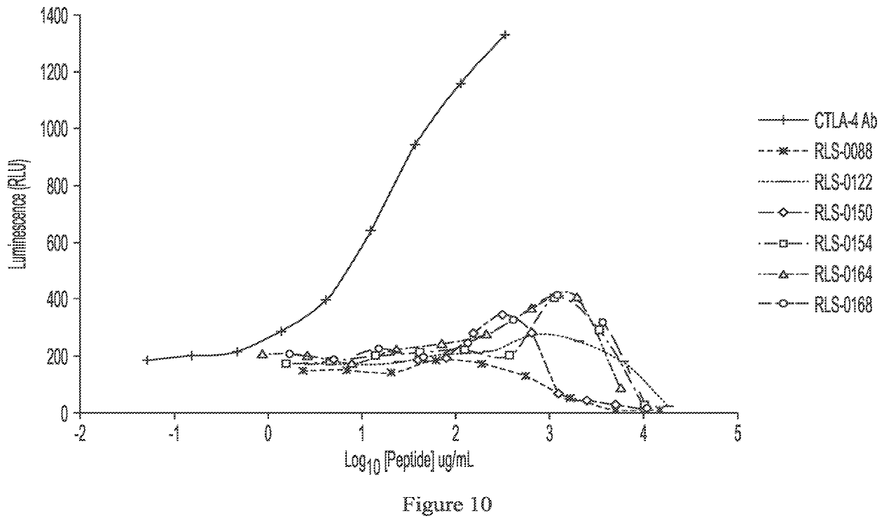

To assess if PIC1 peptides could block CTLA-4 inhibitory activity in a cell-based assay, select peptides were screened in the CTLA-4 Blockade Bioassay (Promega). This bioluminescent cell-based assay can be used to measure the potency and stability of molecules targeting CTLA-4 and consists of two genetically engineered cell lines: CTLA-4 effector cells: Jurkat T cells expressing human CTLA-4 and a luciferase reporter driven by a native promoter which responds to TCR/CD28 activation and aAPC/Raji Cells: Raji cells expressing an engineered cell surface protein designed to activate cognate TCRs in an antigen-independent manner and endogenously expressing CTLA-4 ligands CD80 and CD86. When the two cell types are co-cultured, CTLA-4 competes with CD28 for their shared ligands, CD80 and CD86 and thus inhibits CD28 pathway activation and promoter-mediated luminescence. Addition of a molecule that blocks the interaction of CTLA-4 with its ligands CD80 and CD86 results in promoter-mediated luminescence. The CTLA-4 antibody, used as a positive control, showed a dose-dependent increase in luminescence indicative of inhibition of CTLA-4 binding to its cognate receptor (FIG. 10). PIC1 peptides RLS-0122, RLS-0150, RLS-0154, RLS-0164 and RLS-0168 all showed inhibitory activity in the assay as shown by the increase in signal whereas RLS-0088 (negative control) showed no inhibitory over background in the blockade bioassay. These data are summarized in Table 4 and demonstrate that PIC1 peptides can inhibit CTLA-4 interaction with its cognate receptors and can functionally inhibit CTLA-4 mediated signaling in a cell-based bioassay.

TABLE 4
PIC1 peptides and summary of properties

| | SEQ | | | | T-cell | T-Cell | T-Cell | | | Angio- |
|---|---|---|---|---|---|---|---|---|---|---|
| | ID | PD-1 | CTLA-4 | CTLA-4 | Caspase | IL-2 | IFN-γ | VEGF | VEGF | genesis |
| Name | NO. | Binding | Binding | Bioassay | inhibition | secretion | secretion | Binding | Bioassay | inhibition |
| RLS- | 6 | ND | ND | + | ++++ | ++ | ++ | +++ | − | + |
| 0122 | | | | | | | | | | |
| RLS- | 7 | ++++* | ++ | ND | ND | ND | ND | ND | ND | ND |
| 0134 | | | | | | | | | | |
| RLS- | 8 | +++* | ND | ND | ND | ND | ND | ND | ND | ND |
| 0142 | | | | | | | | | | |
| RLS- | 9 | ++++* | ++++ | + | − | − | ++++ | + | ++ | +++ |
| 0150 | | | | | | | | | | |
| RLS- | 10 | ++++* | ND | ND | ND | ND | ND | ND | ND | ND |
| 0152 | | | | | | | | | | |
| RLS- | 11 | ++++* | ND | +/− | − | ++ | − | + | + | +++ |
| 0154 | | | | | | | | | | |
| RLS- | 12 | ND | ND | +/− | ++++ | +++ | ++++ | ++++ | − | ++++ |
| 0164 | | | | | | | | | | |
| RLS- | 13 | + | ND | + | ++++ | ND | + | + | − | ++++ |
| 0168 | | | | | | | | | | |

### Example 5. Inhibition of T Cell Exhaustion by PIC1 Peptides

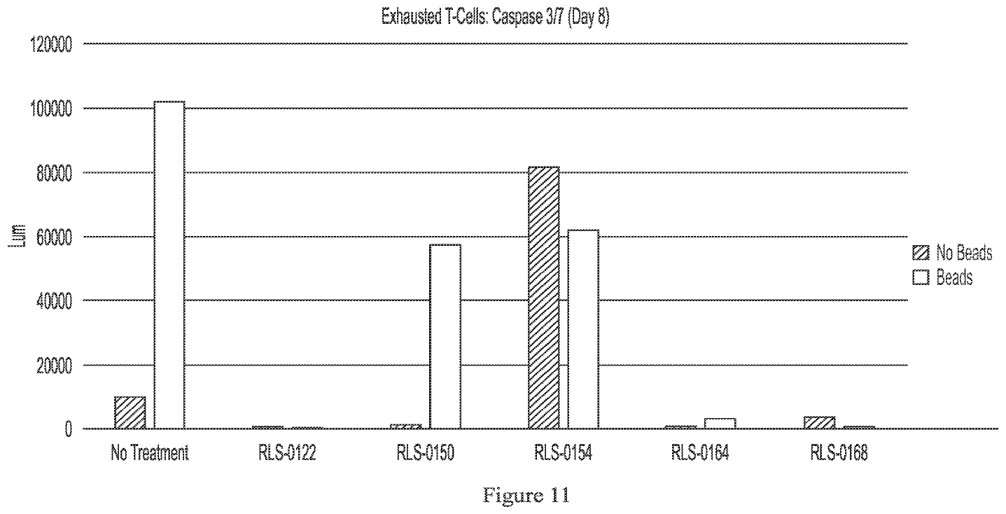

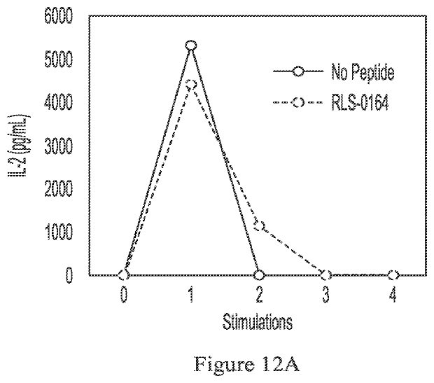

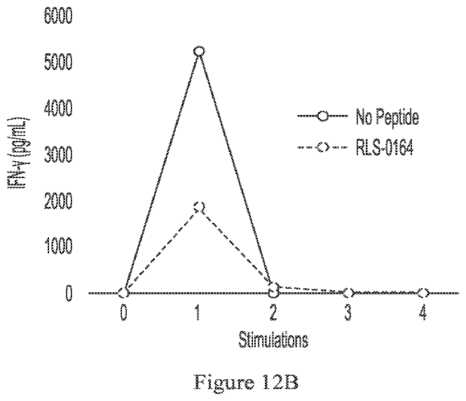

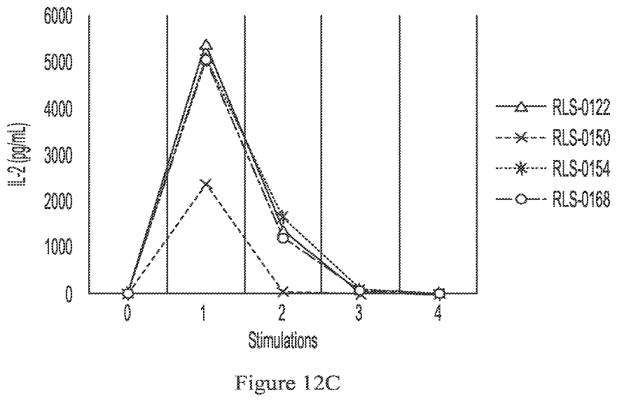

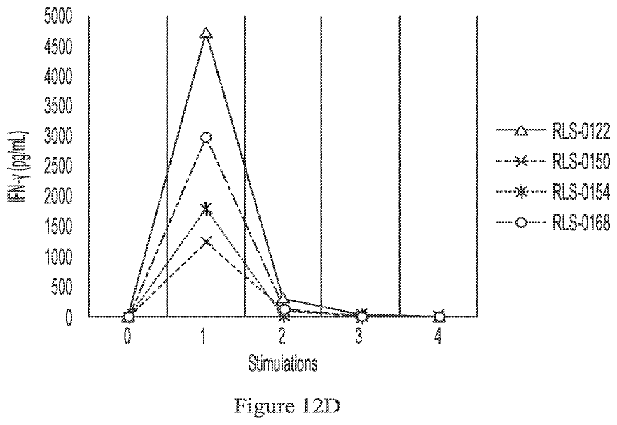

T-cell exhaustion is a form of T-cell dysfunction that occurs in cancer. It is generally defined by poor effector function, reduction in cytokine release (e.g., IL-2, TNF-alpha, IFN-gamma), sustained expression of inhibitory receptors (e.g., PD-1, LAG-3, CD244, CD160) with the progressive loss of effector function due to overstimulation. Exhaustion can prevent optimal control of tumor growth. Exhausted T cells are prevalent in the tumor microenvironment (TME) and can lead to T-cell apoptosis. T-cell exhaustion is reversible and pharmaceutical inhibition of T-cell exhaustion has been considered a promising strategy by many cancer researchers. To ascertain if PIC1 peptides could reverse T-cell exhaustion and increase T cell viability and effector function, a T-cell exhaustion protocol was developed. To induce T-cell exhaustion, purified human Pan T-cells were stimulated with T-Activator CD3/CD28 Dynabeads and cells were washed and re-stimulated every 48 hours. PIC1 peptides were added to the cells after each stimulation with Dynabeads. After three to four stimulations, cells were harvested for readouts which consisted of assessing T-cell apoptosis by measuring Caspase 3/7 levels and production of cytokines IL-2 and IFN-gamma, which are indicative of T-cell functionality. T-cells receiving bead stimulation and not treated with peptide showed an increase in Caspase 3/7 levels indicative of apoptosis, whereas unstimulated cells showed low levels of Caspase 3/7 signal (No treatment, FIG. 11). T-cells treated with PIC1 peptides RLS-0122, RLS-0164 and RLS-0168 showed decreased levels of Caspase 3/7 with some peptides such as RLS-0122 and RLS-0168 showing very low to undetectable levels of Caspase 3/7. In contrast, RLS-0150 and RLS-0154 had an increase in Caspase 3/7 levels. To further evaluate if PIC1 peptides could restore T-cell functionality in cells that have undergone the exhaustion protocol, the inventors next assessed production of cytokines IL-2 and IFN-gamma. Supernatants from the cells after each stimulation was collected and the cytokines measured by ELISA. As shown in FIG. 12A-12B, cells receiving no peptide had a spike of IL-2 or IFN-gamma signal at Dynabead stimulation 1 which was then not detectable by stimulation 2. In contrast, cells treated with RLS-0164 showed IL-2 and IFN-gamma signal at stimulation 2. The lower level of IFN-gamma signal was consistently seen in this assay. Compared to RLS-0150, which does not inhibit T-cell exhaustion, RLS-0122, RLS-0154 and RLS-0168 all showed detectable IL-2 at stimulation 2 (FIG. 12C). IFN-gamma signal was detectable at stimulation 2 for RLS-0122 and RLS-0168, but not RLS-0154 (FIG. 12D). These data are summarized in Table 4 and demonstrate that PIC1 peptides can inhibit human T-cell exhaustion as measured by inhibition of apoptotic markers and restoration of cytokine production.

### Example 6. Binding VEGF and Inhibition of VEGF Function by PIC1 Peptides

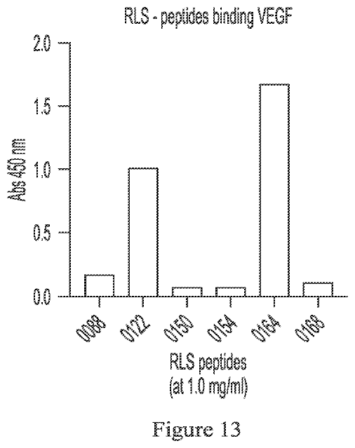

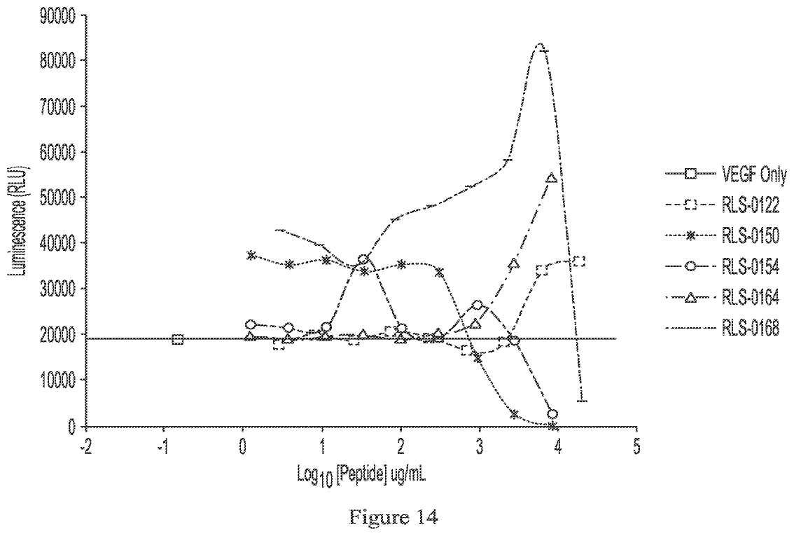

Angiogenesis, or the formation of new blood vessels from the established vasculature, is an essential element in tumor growth and metastasis formation. Inhibiting tumor angiogenesis is considered a major therapeutic strategy in oncology. Vascular endothelial growth factor (VEGF) is a potent and specific angiogenic factor and is a key requirement for tumor growth. VEGF inhibitors, such as monoclonal antibodies, are currently utilized to inhibit tumor growth in cancer patients. Although these anti-VEGF medications have proven to be effective for late-stage and metastatic cancers, they have been demonstrated to cause side effects such as hypertension, artery clots, complications in wound healing, and, more rarely, gastrointestinal perforation and fistulas. Thus, there is a need for safe VEGF inhibitors not based on monoclonal antibody technology. The inventors tested whether these PIC1 peptides possess the ability to bind human VEGF and inhibit VEGF function in a cell-based bioassay. To ascertain the ability of PIC1 peptides to bind VEGF, a binding assay was performed in which VEGF was coated on a microtiter plate followed by incubation with PIC1 peptides (1 mg/ml). As shown in FIG. 13, PIC1 peptides bound VEGF at varying levels, with RLS-0122 and RLS-0164 binding with high affinity. Next, the inventors determined whether these PIC1 peptides could functionally inhibit VEGF mediated cell signaling via its cognate cell surface receptor, VEGFR-2 (KDR) using a VEGF bioassay (Promega). The VEGF Bioassay is a bioluminescent cell-based assay that measures VEGF stimulation and inhibition of VEGFR-2 using luciferase as a readout. This assay can be used for discovery and development of novel biologic therapies aimed at either inducing or inhibiting the VEGF response. The VEGF responsive cells have been engineered to express the response element (RE) upstream of luc2P, as well as exogenous VEGF receptor. When VEGF binds to VEGF responsive cells, the receptor transduces intracellular signals, resulting in luminescence. The bioluminescent signal is detected in a luminometer. As shown in FIG. 14, addition of VEGF incubated with cells without peptide gave a signal of 20,000 relative luminescence units (RLS) response, which is indicative of VEGF binding VEGFR-2. RLS-0150 and RLS-0154 dose-dependently reduced luminescence, indicative of blocking VEGF signaling via VEGFR-2, whereas RLS-0122, RLS-0164 and RLS-0168 did not inhibit signaling. These data are summarized in Table 4.

### Example 7. Inhibition of Non-VEGF Mediated Angiogenesis by PIC1 Peptides

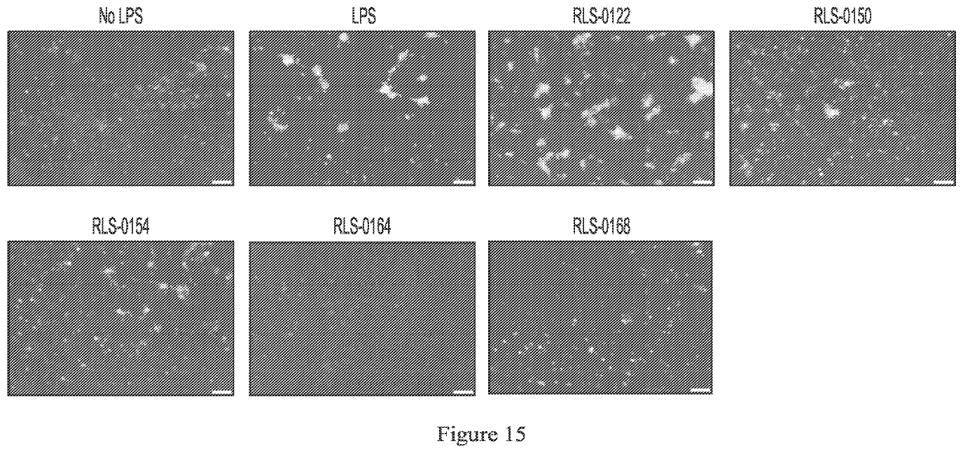

While VEGF plays a major role in angiogenesis in cancer, other non-VEGF factors can induce angiogenesis to promote tumor growth. Currently there are no drugs on the market to inhibit non-VEGF mediated angiogenesis. To assess whether the PIC1 peptides could inhibit non-VEGF mediated angiogenesis, the inventors developed a model of angiogenesis using human umbilical endothelial vein cells (HUVECs) in which addition of lipopolysaccharide (LPS) induces angiogenesis. HUVECs were first incubated with Cell Trace Violet dye, followed by addition of PIC1 peptides (10 mg/ml) for 1 hour at 37° C. and then treated with 10 ug/ml of LPS and placement on an extracellular matrix to promote angiogenesis. Cells were incubated overnight at 37° C. in a humidified CO₂ incubator. Cells were then visualized for tube formation indicative of angiogenesis by fluorescence microscopy. Cells not receiving LPS did not show any clumping or formation of tube buds whereas these structures were evident in cells treated with LPS (FIG. 15). In the presence of PIC1 peptides, varying levels of angiogenesis inhibition were seen, with some peptides (RLS-0164 and RLS-0168) showing no detectable tube formation similar to control cells not stimulated with LPS. These data are summarized in Table 4.

### Example 8. Inhibition of Complement Activation in Human Kidney Carcinoma Cell Line A498 by RLS-0122

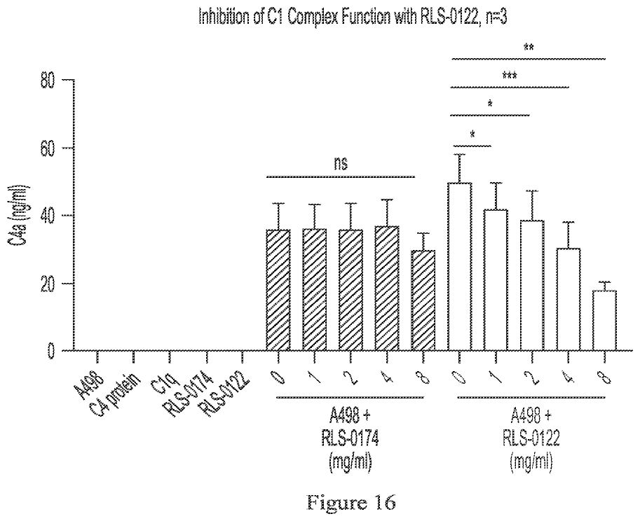

Effective clear-cell renal cell carcinoma (ccRCC) therapies are needed, particularly at the metastatic stage when surgery is ineffective. Complement is a key factor in tissue inflammation, favoring cancer progression through the production of complement component 5a (C5a) (Roumenina et al., Cancer Immunol Res; 7(7) July 2019). Using data mining techniques, ccRCC has been identified as a cancer type expressing concomitantly high expression of the components that are part of the classical complement pathway. High densities of cells producing classical complement pathway components C1q and C4 and the presence of C4 activation fragment deposits in primary tumors are often correlated with poor prognosis. Thus, inhibition of the classical complement pathway may present a novel therapeutic strategy to potentially reduce tumor growth in ccRCC and other cancers in which complement activation plays a tumor promoting inflammatory role such as lung cancer as well as head and neck squamous cell carcinoma (HNSCC). The inventors tested the ability of RLS-0122 to block complement activation in human kidney carcinoma cell line A498 system in vitro as assessed by the reduction in C4a levels. The A-498 cell line, derived from a human kidney carcinoma, produces complement component products C1r and C1s. The addition of purified C1q creates a functional C1 complex (Roumenina et al., Cancer Immunol Res; 7(7) July 2019). RLS-0122 was chosen to be evaluated in this in vitro assay based on its ability to potently block complement activation in a hemolytic assay (FIG. 1). Concentrated supernatant from serum starved A-498 cells (3%) were added to purified human C1q (0.5 ug/ml) pre-incubated with increasing doses (0-8 mg/ml) of RLS-0122 or RLS-0174 (negative control peptide) and loaded onto an IgG coated plate. Samples were then incubated for 1 hour at 37° C. After incubation, samples were washed 3 times with PBS-T before the addition of purified human C4 (4 ug/ml) and incubated for 1.5 hours at 37° C. The supernatants were collected for analysis on Quidel's MicroVue Complement C4a ELISA. RLS-0122 dose-dependently reduced the level of C4a generation, whereas a peptide that does not inhibit classical complement activity (RLS-0174) had no significant inhibitory activity (FIG. 16).

### Example 9. RLS-0122 Increases Survival and Quality of Life in a Mouse TC-1 Tumor Cell Model

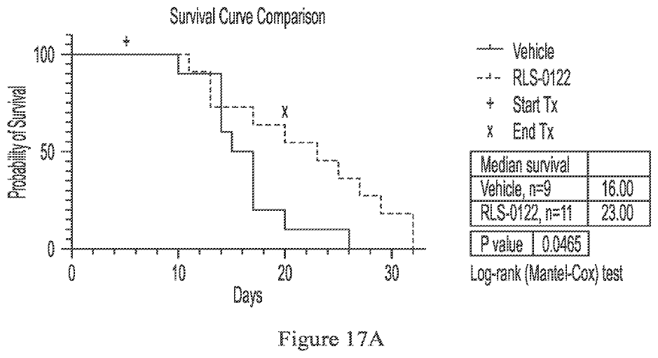

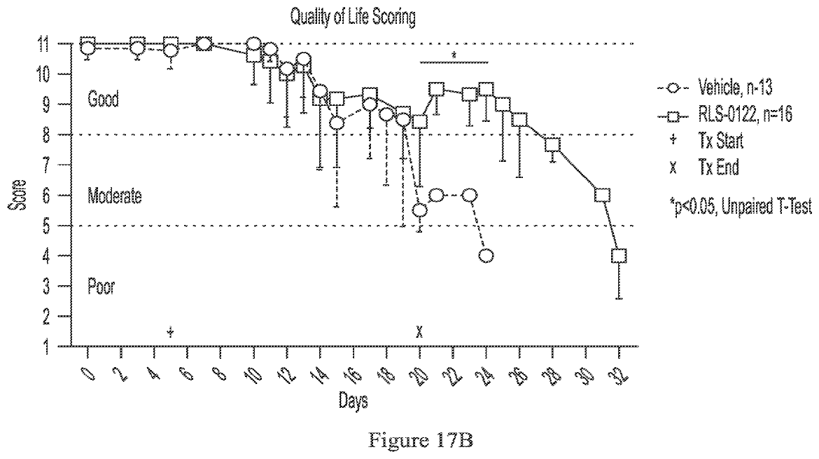

To evaluate the efficacy of RLS-0122 in an in vivo system, the inventors utilized the TC-1 tumor cell model in C57Bl/6 mice as previously reported (Roumenina et al., Cancer Immunol Res; 7(7) July 2019). TC-1 cells are derived from a lung epithelial cell line transformed with human papillomavirus where complement activation has been demonstrated to contribute to tumor growth. When TC-1 cells are introduced into the flank of the mouse, very aggressive tumor formation occurs, resulting in 100% death in approximately 26 days. To test the efficacy of RLS-0122 on survival and quality of life in this model, animals were injected with 4×10\hat{ }5 TC-1 cells subcutaneously into the flank of the animal. Five days later, animals received vehicle treatment with saline (n=6), or drug treatment with RLS-0122 (n=8) IV at 160 mg/kg 1×/day for 15 consecutive days. Animals treated with RLS-0122 displayed a significant median survival of 8 days longer than those who received vehicle (p=0.0465) (FIG. 17A). Animals were also evaluated for behavior and body condition scoring every 2 days for duration of the study. If scoring reached 7 or below, animals were assessed daily. A score of 5 or below was considered endpoint for euthanasia. Animals receiving RLS-0122 demonstrated increased quality of life after end of treatment with scores reaching statistical significance on days 22-26 (p<0.05) (FIG. 17A-17B).

### Example 10: Administration of Pharmaceutical Compositions

A pharmaceutical composition comprising a therapeutically effective amount of any of SEQ ID NOs: 6-13, and variants thereof, is administered to a subject in need thereof to regulate the complement system.

A pharmaceutical composition comprising a therapeutically effective amount of any of SEQ ID NOs: 6-13, and variants thereof, is administered to a subject in need thereof to inhibit myeloperoxidase activity.

A pharmaceutical composition comprising a therapeutically effective amount of any of SEQ ID NOs: 6-13, and variants thereof, is administered to a subject in need thereof to inhibit oxidant activity.

A pharmaceutical composition comprising a therapeutically effective amount of any of SEQ ID NOs: 6-13, and variants thereof, is administered to a subject in need thereof to inhibit PD-1 binding to PD-L1.

A pharmaceutical composition comprising a therapeutically effective amount of any of SEQ ID NOs: 6-13, and variants thereof, is administered to a subject in need thereof to inhibit T cell exhaustion.

A pharmaceutical composition comprising a therapeutically effective amount of any of SEQ ID NOs: 6-13, and variants thereof, is administered to a subject in need thereof to inhibiting angiogenesis.

### LIST OF EMBODIMENTS

The following is a non-exhaustive list of embodiments provided by the invention:
1. A synthetic peptide comprising at least about 95% sequence identity to an amino acid sequence selected from the group of SEQ ID NO: 6-13.
2. The synthetic peptide of embodiment 1 comprising an amino acid sequence selected from the group consisting of SEQ ID NO: 6-13.
3. The synthetic peptide of embodiment 1 comprising at least about 95% sequence identity to amino acid sequence selected from the group consisting of SEQ ID NOs: 7-11.
4. The synthetic peptide of embodiment 1 comprising an amino acid sequence selected from the group consisting of SEQ ID NOs: 7-11.
5. The synthetic peptide of claim 1 consisting of an amino acid sequence comprising at least about 95% sequence identity to SEQ ID NO: 6.
6. The synthetic peptide of claim 1 consisting of an amino acid sequence comprising at least about 95% sequence identity to SEQ ID NO: 7.
7. The synthetic peptide of claim 1 consisting of an amino acid sequence comprising at least about 95% sequence identity to SEQ ID NO: 8.
8. The synthetic peptide of claim 1 consisting of an amino acid sequence comprising at least about 95% sequence identity to SEQ ID NO: 9.
9. The synthetic peptide of claim 1 consisting of an amino acid sequence comprising at least about 95% sequence identity to SEQ ID NO: 10.
10. The synthetic peptide of claim 1 consisting of an amino acid sequence comprising at least about 95% sequence identity to SEQ ID NO: 11.
11. The synthetic peptide of claim 1 consisting of an amino acid sequence comprising at least about 95% sequence identity to SEQ ID NO: 12.
12. The synthetic peptide of claim 1 consisting of an amino acid sequence comprising at least about 95% sequence identity to SEQ ID NO: 13.
13. A pharmaceutical composition comprising a therapeutically effective amount of the synthetic peptide of any of claims 1-12 and at least one pharmaceutically acceptable carrier, diluent, or excipient.
14. A method of regulating the complement system comprising administering the pharmaceutical composition of claim 13 to a subject in need thereof.

15. A method of inhibiting myeloperoxidase activity comprising administering the pharmaceutical composition of claim 13 to a subject in need thereof.

16. A method of inhibiting oxidant activity comprising administering the pharmaceutical composition of claim 13 to a subject in need thereof.

17. A method of inhibiting PD-1 binding to PD-L1 comprising administering the pharmaceutical composition of claim 13 to a subject in need thereof.

18. A method of inhibiting T cell exhaustion comprising administering the pharmaceutical composition of claim 13 to a subject in need thereof.

19. A method of inhibiting angiogenesis comprising administering the pharmaceutical composition of claim 13 to a subject in need thereof.

Sequence Table

| SEQ | | |
|---|---|---|
| ID | | |
| NO. | Sequence | Description |
| 1 | PAICQRATATLGTVGSNTSGTTEIEACILL | Astrovirus CP protein |
| 2 | IALILEPICCQERAA | PA (PIC1) |
| 3 | IALILEPICCQERAA-PEG24 | PA-dPEG24 |
| 4 | IALILEP(Sar)CCQERAA | PA-I8Sar; RLS-0088 |
| 5 | IALILEPI(Sar)CQERAA | PA-C9Sar; RLS-0089 |
| 6 | IAdLILEP(Sar)CCQERAA | PA-0122 (RLS-0122), D-enantiomer |
| | | substitution of SEQ ID NO: 4 |
| 7 | IAR8ILEP(Sar)CS5QERAA | PA-0134 (RLS-0134), stapled |
| | | modification of SEQ ID NO: 4 |
| 8 | IAdLILEP(Sar)CS5QERS5A | PA-0142 (RLS-0142), stapled and D- |
| | | enantiomeric modification of SEQ ID NO: |
| | | 4 |
| 9 | IALILER8I(Sar)CQERS5A | PA-0150 (RLS-0150), stapled |
| | | modification of SEQ ID NO: 5 |
| 10 | IS5LILS5PI(Sar)CQERAA | PA-0152 (RLS-0152), stapled |
| | | modification of SEQ ID NO: 5 |
| 11 | IALILES5I(Sar)CS5ERAA | PA-0154 (RLS-0154), stapled |
| | | modification of SEQ ID NO: 5 |
| 12 | IALILEPI(Sar)dCQERAA | PA-0164 (RLS-0164), D-enantiomeric |
| | | modification of SEQ ID NO: 5 |
| 13 | IALILEPI(Sar)CQERdAA | PA-0168 (RLS-0168), D-enantiomeric |
| | | modification of SEQ ID NO: 5 |

While several possible embodiments are disclosed above, embodiments of the present invention are not so limited. These exemplary embodiments are not intended to be exhaustive or to unnecessarily limit the scope of the invention, but instead were chosen and described in order to explain the principles of the present invention so that others skilled in the art may practice the invention. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

### REFERENCES

1. Fosgerau K and Hoffmann T (2014) Peptide therapeutics: current status and future directions. Drug Disc Today 20: 122-129.
2. Ali A M, Atmaj J, Van Oosterwijk N, Groves M R, Domling A (2019) Stapled peptide inhibitors: a new window for target drug discovery. Comp Struct Biotech J 17: 263-281
3. Sharp J A, Hair P S, Pallera H K, Kumar P S, Mauriello C T, et al. (2015) Peptide Inhibitor of Complement C1 (PIC1) Rapidly Inhibits Complement Activation after Intravascular Injection in Rats. PLoS ONE 10: e0132446.
4. Hair P S, Sass L A, Krishna N K, Cunnion K M (2017) Inhibition of Myeloperoxidase Activity in Cystic Fibrosis Sputum by Peptide Inhibitor of Complement C1 (PIC1). PLoS ONE 12: e0170203.
5. Hair P S, Cunnion K M, Krishna N K (2017) Peptide Inhibitor of Complement C1 Inhibits the Peroxidase Activity of Hemoglobin and Myoglobin. Int J Pept 2017: 9454583.
6. Gregory Rivera M, Hair P S, Cunnion K M, Krishna N K (2018) Peptide Inhibitor of Complement C1 (PIC1) demonstrates antioxidant activity via single electron transport (SET) and hydrogen atom transfer (HAT). PLoS ONE 13: e0193931.
7. Hair P S, Enos A I, Krishna N K, Cunnion K M (2018) Inhibition of Immune Complex Complement Activation and Neutrophil Extracellular Trap Formation by Peptide Inhibitor of Complement C1. Front Immunol 9: 558.
8. Hair P S, Rivera M G, Enos A I, Pearsall S E, Sharp J A, et al. (2017) Peptide Inhibitor of Complement C1 (PIC1) Inhibits Growth of Pathogenic Bacteria. International Journal of Peptide Research and Therapeutics DOI 101007/s10989-017-9651-z.
9. Matsui S M, Kiang D, Ginzton N, Chew T, Geigenmuller-Gnirke U (2001) Molecular biology of astroviruses: selected highlights. Novartis Found Symp 238: 219-233; discussion 233-216.
10. Bonaparte R S, Hair P S, Banthia D, Marshall D M, Cunnion K M, et al. (2008) Human astrovirus coat protein inhibits serum complement activation via C1, the first component of the classical pathway. J Virol 82: 817-827.
11. Hair P S, Enos A I, Krishna N K, Cunnion K M. (2019) Inhibition of complement activation, myeloperoxidase, NET formation and oxidant activity by PIC1 peptide variants. PLoS ONE 14: e0226875.
12. Cunnion K M, Lee J C, Frank M M (2001) Capsule production and growth phase influence binding of complement to Staphylococcus aureus. Infect Immun 69: 6796-6803.
13. Mauriello C T, Pallera H K, Sharp J A, Woltmann J L, Jr., Qian S, et al. (2013) A novel peptide inhibitor of classical and lectin complement activation including ABO incompatibility. Mol Immunol 53: 132-139.
14. Carlin J B, Doyle L W (2001) Statistics for clinicians: 4: Basic concepts of statistical reasoning: hypothesis tests and the t-test. J Paediatr Child Health 37: 72-77.
15. Rai J (2019) Peptide and Protein Mimetics by Retro and Retroinverso Analogs. Chem Biol Drug Des 93:7 24-736.

1. A synthetic peptide comprising at least about 95% sequence identity to an amino acid sequence selected from the group of SEQ ID NO: 6-13.

2. The synthetic peptide of claim 1 comprising an amino acid sequence selected from the group consisting of SEQ ID NO: 6-13.

3. The synthetic peptide of claim 1 comprising at least about 95% sequence identity to amino acid sequence selected from the group consisting of SEQ ID NOs: 7-11.

4. The synthetic peptide of claim 1 comprising an amino acid sequence selected from the group consisting of SEQ ID NOs: 7-11.

5. The synthetic peptide of claim 1 consisting of an amino acid sequence comprising at least about 95% sequence identity to SEQ ID NO: 6.

6. The synthetic peptide of claim 1 consisting of an amino acid sequence comprising at least about 95% sequence identity to SEQ ID NO: 7.

7. The synthetic peptide of claim 1 consisting of an amino acid sequence comprising at least about 95% sequence identity to SEQ ID NO: 8.

8. The synthetic peptide of claim 1 consisting of an amino acid sequence comprising at least about 95% sequence identity to SEQ ID NO: 9.

9. The synthetic peptide of claim 1 consisting of an amino acid sequence comprising at least about 95% sequence identity to SEQ ID NO: 10.

10. The synthetic peptide of claim 1 consisting of an amino acid sequence comprising at least about 95% sequence identity to SEQ ID NO: 11.

11. The synthetic peptide of claim 1 consisting of an amino acid sequence comprising at least about 95% sequence identity to SEQ ID NO: 12.

12. The synthetic peptide of claim 1 consisting of an amino acid sequence comprising at least about 95% sequence identity to SEQ ID NO: 13.

13. A pharmaceutical composition comprising a therapeutically effective amount of the synthetic peptide of claim 1 and at least one pharmaceutically acceptable carrier, diluent, or excipient.

14. A method of regulating the complement system comprising administering the pharmaceutical composition of claim 13 to a subject in need thereof.

15. A method of inhibiting myeloperoxidase activity comprising administering the pharmaceutical composition of claim 13 to a subject in need thereof.

16. A method of inhibiting oxidant activity comprising administering the pharmaceutical composition of claim 13 to a subject in need thereof.

17. A method of inhibiting PD-1 binding to PD-L1 comprising administering the pharmaceutical composition of claim 13 to a subject in need thereof.

18. A method of inhibiting T cell exhaustion comprising administering the pharmaceutical composition of claim 13 to a subject in need thereof.

19. A method of inhibiting angiogenesis comprising administering the pharmaceutical composition of claim 13 to a subject in need thereof.
